SEQUENCE LISTING (20 sequences)

SEQ ID NO: 1 - DNA, 1419 bp, Homo sapiens
atgttaggaactgtgaagatggaagggcatgaaaccagcgactggaacagctactacgcagacacgcaggaggcctactcctccgtcccggtcagcaacatgaactcaggcctgggctccatgaactccatgaacacctacatgaccatgaacaccatgactacgagcggcaacatgaccccggcgtccttcaacatgtcctatgccaacccgggcctaggggccggcctgagtcccggcgcagtagccggcatgccggggggctcggcgggcgccatgaacagcatgactgcggccggcgtgacggccatgggtacggcgctgagcccgagcggcatgggcgccatgggtgcgcagcaggcggcctccatgaatggcctgggcccctacgcggccgccatgaacccgtgcatgagccccatggcgtacgcgccgtccaacctgggccgcagccgcgcgggcggcggcggcgacgccaagacgttcaagcgcagctacccgcacgccaagccgccctactcgtacatctcgctcatcaccatggccatccagcaggcgcccagcaagatgctcacgctgagcgagatctaccagtggatcatggacctcttcccctattaccggcagaaccagcagcgctggcagaactccatccgccactcgctgtccttcaatgactgcttcgtcaaggtggcacgctccccggacaagccgggcaagggctcctactggacgctgcacccggactccggcaacatgttcgagaacggctgctacttgcgccgccagaagcgcttcaagtgcgagaagcagccgggggccggcggcgggggcgggagcggaagcgggggcagcggcgccaagggcggccctgagagccgcaaggacccctctggcgcctctaaccccagcgccgactcgcccctccatcggggtgtgcacgggaagaccggccagctagagggcgcgccggcccccgggcccgccgccagcccccagactctggaccacagtggggcgacggcgacagggggcgcctcggagttgaagactccagcctcctcaactgcgccccccataagctccgggcccggggcgctggcctctgtgcccgcctctcacccggcacacggcttggcaccccacgagtcccagctgcacctgaaaggggacccccactactccttcaaccacccgttctccatcaacaacctcatgtcctcctcggagcagcagcataagctggacttcaaggcatacgaacaggcactgcaatactcgccttacggctctacgttgcccgccagcctgcctctaggcagcgcctcggtgaccaccaggagccccatcgagccctcagccctggagccggcgtactaccaaggtgtgtattccagacccgtcctaaacacttcctag

SEQ ID NO: 2 - protein, 472 aa, Homo sapiens
MLGTVKMEGHETSDWNSYYADTQEAYSSVPVSNMNSGLGSMNSMNTYMTMNTMTTSGNMTPASFNMSYANPGLGAGLSPGAVAGMPGGSAGAMNSMTAAGVTAMGTALSPSGMGAMGAQQAASMNGLGPYAAAMNPCMSPMAYAPSNLGRSRAGGGGDAKTFKRSYPHAKPPYSYISLITMAIQQAPSKMLTLSEIYQWIMDLFPYYRQNQQRWQNSIRHSLSFNDCFVKVARSPDKPGKGSYWTLHPDSGNMFENGCYLRRQKRFKCEKQPGAGGGGGSGSGGSGAKGGPESRKDPSGASNPSADSPLHRGVHGKTGQLEGAPAPGPAASPQTLDHSGATATGGASELKTPASSTAPPISSGPGALASVPASHPAHGLAPHESQLHLKGDPHYSFNHPFSINNLMSSSEQQHKLDFKAYEQALQYSPYGSTLPASLPLGSASVTTRSPIEPSALEPAYYQGVYSRPVLNTS

SEQ ID NO: 3 - DNA, 1392 bp, Homo sapiens
atgcactcggcttccagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaa

SEQ ID NO: 4 - protein, 463 aa, Homo sapiens
MHSASSMLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 5 - DNA, 1053 bp, Homo sapiens
atgctgggctcagtgaagatggaggcccatgacctggccgagtggagctactacccggaggcgggcgaggtctactcgccggtgaccccagtgcccaccatggcccccctcaactcctacatgaccctgaatcctctaagctctccctatccccctggggggctccctgcctccccactgccctcaggacccctggcacccccagcacctgcagcccccctggggcccactttcccaggcctgggtgtcagcggtggcagcagcagctccgggtacggggccccgggtcctgggctggtgcacgggaaggagatgccgaaggggtatcggcggcccctggcacacgccaagccaccgtattcctatatctcactcatcaccatggccatccagcaggcgccgggcaagatgctgaccttgagtgaaatctaccagtggatcatggacctcttcccttactaccgggagaatcagcagcgctggcagaactccattcgccactcgctgtctttcaacgactgcttcgtcaaggtggcgcgttccccagacaagcctggcaagggctcctactgggccctacaccccagctcagggaacatgtttgagaatggctgctacctgcgccgccagaaacgcttcaagctggaggagaaggtgaaaaaagggggcagcggggctgccaccaccaccaggaacgggacagggtctgctgcctcgaccaccacccccgcggccacagtcacctccccgccccagcccccgcctccagcccctgagcctgaggcccagggcggggaagatgtgggggctctggactgtggctcacccgcttcctccacaccctatttcactggcctggagctcccaggggagctgaagctggacgcgccctacaacttcaaccaccctttctccatcaacaacctaatgtcagaacagacaccagcacctcccaaactggacgtggggtttgggggctacggggctgaaggtggggagcctggagtctactaccagggcctctattcccgctctttgcttaatgcatcctag

SEQ ID NO: 6 - protein, 350 aa, Homo sapiens
MLGSVKMEAHDLAEWSYYPEAGEVYSPVTPVPTMAPLNSYMTLNPLSSPYPPGGLPASPLPSGPLAPPAPAAPLGPTFPGLGVSGGSSSSGYGAPGPGLVHGKEMPKGYRRPLAHAKPPYSYISLITMAIQQAPGKMLTLSEIYQWIMDLFPYYRENQQRWQNSIRHSLSFNDCFVKVARSPDKPGKGSYWALHPSSGNMFENGCYLRRQKRFKLEEKVKKGGSGAATTTRNGTGSAASTTTPAATVTSPPQPPPPAPEPEAQGGEDVGALDCGSPASSTPYFTGLELPGELKLDAPYNFNHPFSINNLMSEQTPAPPKLDVGFGGYGAEGGEPGVYYQGLYSRSLLNAS

SEQ ID NO: 7 - DNA, 1077 bp, Homo sapiens
atggagtcggccgacttctacgaggcggagccgcggcccccgatgagcagccacctgcagagccccccgcacgcgcccagcagcgccgccttcggctttccccggggcgcgggccccgcgcagcctcccgccccacctgccgccccggagccgctgggcggcatctgcgagcacgagacgtccatcgacatcagcgcctacatcgacccggccgccttcaacgacgagttcctggccgacctgttccagcacagccggcagcaggagaaggccaaggcggccgtgggccccacgggcggcggcggcggcggcgactttgactacccgggcgcgcccgcgggccccggcggcgccgtcatgcccgggggagcgcacgggcccccgcccggctacggctgcgcggccgccggctacctggacggcaggctggagcccctgtacgagcgcgtcggggcgccggcgctgcggccgctggtgatcaagcaggagccccgcgaggaggatgaagccaagcagctggcgctggccggcctcttcccttaccagccgccgccgccgccgccgccctcgcacccgcacccgcacccgccgcccgcgcacctggccgccccgcacctgcagttccagatcgcgcactgcggccagaccaccatgcacctgcagcccggtcaccccacgccgccgcccacgcccgtgcccagcccgcaccccgcgcccgcgctcggtgccgccggcctgccgggccctggcagcgcgctcaaggggctgggcgccgcgcaccccgacctccgcgcgagtggcggcagcggcgcgggcaaggccaagaagtcggtggacaagaacagcaacgagtaccgggtgcggcgcgagcgcaacaacatcgcggtgcgcaagagccgcgacaaggccaagcagcgcaacgtggagacgcagcagaaggtgctggagctgaccagtgacaatgaccgcctgcgcaagcgggtggaacagctgagccgcgaactggacacgctgcggggcatcttccgccagctgccagagagctccttggtcaaggccatgggcaactgcgcgtga

SEQ ID NO: 8 - protein, 358 aa, Homo sapiens
MESADFYEAEPRPPMSSHLQSPPHAPSSAAFGFPRGAGPAQPPAPPAAPEPLGGICEHETSIDISAYIDPAAFNDEFLADLFQHSRQQEKAKAAVGPTGGGGGGDFDYPGAPAGPGGAVMPGGAHGPPPGYGCAAAGYLDGRLEPLYERVGAPALRPLVIKQEPREEDEAKQLALAGLFPYQPPPPPPPSHPHPHPPPAHLAAPHLQFQIAHCGQTTMHLQPGHPTPPPTPVPSPHPAPALGAAGLPGPGSALKGLGAAHPDLRASGGSGAGKAKKSVDKNSNEYRVRRERNNIAVRKSRDKAKQRNVETQQKVLELTSDNDRLRKRVEQLSRELDTLRGIFRQLPESSLVKAMGNCA

SEQ ID NO: 9 - DNA, 1425 bp, Homo sapiens
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 10 - protein, 474 aa, Homo sapiens
MRLSKTLVDMDMADYSAALDPAYTTLEFENVQVLTMGNDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMCEWPRPRGQAATPETPQPSPPGGSGSEPYKLLPGAVATIVKPLSAIPQPTITKQEVI

SEQ ID NO: 11 - DNA, 1407 bp, Mus musculus
atgttagggactgtgaagatggaagggcatgagagcaacgactggaacagctactacgcggacacgcaggaggcctactcctctgtccctgtcagcaacatgaactccggcctgggctctatgaactccatgaacacctacatgaccatgaacaccatgaccacgagcggcaacatgaccccggcttccttcaacatgtcctacgccaacacgggcttaggggccggcctgagtcccggtgctgtggctggcatgccaggggcctctgcaggcgccatgaacagcatgactgcggcgggcgtcacggccatgggtacggcgctgagcccgggaggcatgggctccatgggcgcgcagcccgccacctccatgaacggcctgggtccctacgccgccgccatgaacccgtgcatgagtcccatggcgtacgcgccgtccaacctgggccgcagccgcgcggggggcggcggcgacgccaagacattcaagcgcagctaccctcacgccaagccgccttactcctacatctcgctcatcacgatggccatccagcaggcgcccagcaagatgctcacgctgagcgagatctaccagtggatcatggacctcttcccctattaccgccagaaccagcagcgctggcagaactccatccgccactcgctgtccttcaacgattgtttcgtcaaggtggcacgatccccggacaagccaggcaagggctcctactggacgctgcacccggactccggcaacatgttcgagaacggctgctacttgcgccgccaaaagcgcttcaagtgtgagaagcagccgggggccggaggtgggagtgggggcggcggctccaaagggggcccagaaagtcgcaaggacccctcaggcccggggaaccccagcgccgagtcaccccttcaccggggtgtgcacggaaaggctagccagctagagggcgcgccggccccagggcccgccgccagcccccagactctggaccacagcggggccacggcgacagggggcgcttcggagttgaagtctccagcgtcttcatctgcgccccccataagctccgggccaggggcgctagcatctgtacccccctctcacccggctcacggcctggcaccccacgaatctcagctgcatctgaaaggggatccccactactcctttaatcaccccttctccatcaacaacctcatgtcctcctccgagcaacagcacaagctggacttcaaggcatacgagcaggcgctgcagtactctccttatggcgctaccttgcccgccagtctgccccttggcagcgcctcagtggccacgaggagccccatcgagccctcagccctggagccagcctactaccaaggtgtgtattccagacccgtgctaaatacttcctag

SEQ ID NO: 12 - protein, 468 aa, Mus musculus
MLGTVKMEGHESNDWNSYYADTQEAYSSVPVSNMNSGLGSMNSMNTYMTMNTMTTSGNMTPASFNMSYANTGLGAGLSPGAVAGMPGASAGAMNSMTAAGVTAMGTALSPGGMGSMGAQPATSMNGLGPYAAAMNPCMSPMAYAPSNLGRSRAGGGGDAKTFKRSYPHAKPPYSYISLITMAIQQAPSKMLTLSEIYQWIMDLFPYYRQNQQRWQNSIRHSLSFNDCFVKVARSPDKPGKGSYWTLHPDSGNMFENGCYLRRQKRFKCEKQPGAGGGSGGGGSKGGPESRKDPSGPGNPSAESPLHRGVHGKASQLEGAPAPGPAASPQTLDHSGATATGGASELKSPASSSAPPISSGPGALASVPPSHPAHGLAPHESQLHLKGDPHYSFNHPFSINNLMSSSEQQHKLDFKAYEQALQYSPYGATLPASLPLGSASVATRSPIEPSALEPAYYQGVYSRPVLNTS

SEQ ID NO: 13 - DNA, 1380 bp, Mus musculus
atgctgggagccgtgaagatggaagggcacgagccatccgactggagcagctactacgcggagcccgagggctactcttccgtgagcaacatgaacgccggcctggggatgaatggcatgaacacatacatgagcatgtccgcggctgccatgggcggcggttccggcaacatgagcgcgggctccatgaacatgtcatcctatgtgggcgctggaatgagcccgtcgctagctggcatgtccccgggcgccggcgccatggcgggcatgagcggctcagccggggcggccggcgtggcgggcatgggacctcacctgagtccgagtctgagcccgctcgggggacaggcggccggggccatgggtggccttgccccctacgccaacatgaactcgatgagccccatgtacgggcaggccggcctgagccgcgctcgggaccccaagacataccgacgcagctacacacacgccaaacctccctactcgtacatctcgctcatcaccatggccatccagcagagccccaacaagatgctgacgctgagcgagatctatcagtggatcatggacctcttccctttctaccggcagaaccagcagcgctggcagaactccatccgccactctctctccttcaacgactgctttctcaaggtgccccgctcgccagacaagcctggcaagggctccttctggaccctgcacccagactcgggcaacatgttcgagaacggctgctacctgcgccgccagaagcgcttcaagtgtgagaagcaactggcactgaaggaagccgcgggtgcggccagtagcggaggcaagaagaccgctcctgggtcccaggcctctcaggctcagctcggggaggccgcgggctcggcctccgagactccggcgggcaccgagtccccccattccagcgcttctccgtgtcaggagcacaagcgaggtggcctaagcgagctaaagggagcacctgcctctgcgctgagtcctcccgagccggcgccctcgcctgggcagcagcagcaggctgcagcccacctgctgggcccacctcaccacccaggcctgccaccagaggcccacctgaagcccgagcaccattacgccttcaaccaccccttctctatcaacaacctcatgtcgtccgagcagcaacatcaccacagccaccaccaccatcagccccacaaaatggacctcaaggcctacgaacaggtcatgcactacccagggggctatggttcccccatgccaggcagcttggccatgggcccagtcacgaacaaagcgggcctggatgcctcgcccctggctgcagacacttcctactaccaaggagtgtactccaggcctattatgaactcatcctaa

SEQ ID NO: 14 - protein, 459 aa, Mus musculus
MLGAVKMEGHEPSDWSSYYAEPEGYSSVSNMNAGLGMNGMNTYMSMSAAAMGGGSGNMSAGSMNMSSYVGAGMSPSLAGMSPGAGAMAGMSGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAASSGGKKTAPGSQASQAQLGEAAGSASETPAGTESPHSSASPCQEHKRGGLSELKGAPASALSPPEPAPSPGQQQQAAAHLLGPPHHPGLPPEAHLKPEHHYAFNHPFSINNLMSSEQQHHHSHHHHQPHKMDLKAYEQVMHYPGGYGSPMPGSLAMGPVTNKAGLDASPLAADTSYYQGVYSRPIMNSS

SEQ ID NO: 15 - DNA, 1062 bp, Mus musculus
atgctgggctcagtgaagatggaggctcatgacctggccgagtggagctactacccggaggcgggcgaggtgtattctccagtgaatcctgtgcccaccatggcccctctcaactcctacatgaccttgaacccactcagctctccctaccctcccggagggcttcaggcctccccactgcctacaggacccctggcacccccagcccccactgcgcccttggggcccaccttcccaagcttgggcactggtggcagcaccggaggcagtgcttccgggtatgtagccccagggcccgggcttgtacatggaaaagagatggcaaaggggtaccggcggccactggcccacgccaaaccaccatattcctacatctctctcataaccatggctattcagcaggctccaggcaagatgctgaccctgagtgaaatctaccaatggatcatggacctcttcccgtactaccgggagaaccagcaacgttggcagaactccatccggcattcgctgtccttcaatgactgcttcgtcaaggtggcacgctccccagacaagccaggcaaaggctcctactgggccttgcatcccagctctgggaacatgtttgagaacggctgctatctccgccggcagaagcgcttcaagctggaggagaaggcaaagaaaggaaacagcgccacatcggccagcaggaatggtactgcggggtcagccacctctgccaccactacagctgccactgcagtcacctccccggctcagccccagcctacgccatctgagcccgaggcccagagtggggatgatgtggggggtctggactgcgcctcacctccttcgtccacaccttatttcagcggcctggagctcccgggggaactaaagttggatgcgccctataacttcaaccaccctttctctatcaacaacctgatgtcagaacagacatcgacaccttccaaactggatgtggggtttgggggctacggggctgagagtggggagcctggagtctactaccagagcctctattcccgctctctgcttaatgcatcctag

SEQ ID NO: 16 - protein, 353 aa, Mus musculus
MLGSVKMEAHDLAEWSYYPEAGEVYSPVNPVPTMAPLNSYMTLNPLSSPYPPGGLQASPLPTGPLAPPAPTAPLGPTFPSLGTGGSTGGSASGYVAPGPGLVHGKEMAKGYRRPLAHAKPPYSYISLITMAIQQAPGKMLTLSEIYQWIMDLFPYYRENQQRWQNSIRHSLSFNDCFVKVARSPDKPGKGSYWALHPSSGNMFENGCYLRRQKRFKLEEKAKKGNSATSASRNGTAGSATSATTTAATAVTSPAQPQPTPSEPEAQSGDDVGGLDCASPPSSTPYFSGLELPGELKLDAPYNFNHPFSINNLMSEQTSTPSKLDVGFGGYGAESGEPGVYYQSLYSRSLLNAS

SEQ ID NO: 17 - DNA, 1425 bp, Mus musculus
atgcgactctctaaaacccttgccggcatggatatggccgactacagcgctgccctggacccagcctacaccaccctggagtttgaaaatgtgcaggtgttgaccatgggcaatgacacgtccccatctgaaggtgccaacctcaattcatccaacagcctgggcgtcagtgccctgtgcgccatctgtggcgaccgggccaccggcaaacactacggagcctcgagctgtgacggctgcaaggggttcttcaggaggagcgtgaggaagaaccacatgtactcctgcaggtttagccgacaatgtgtggtagacaaagataagaggaaccagtgtcgttactgcaggcttaagaagtgcttccgggctggcatgaagaaggaagctgtccaaaatgagcgggaccggatcagcacgcggaggtcaagctacgaggacagcagcctgccctccatcaacgcgctcctgcaggcagaggttctgtcccagcagatcacctctcccatctctgggatcaatggcgacattcgggcaaagaagattgccaacatcacagacgtgtgtgagtctatgaaggagcagctgctggtcctggtcgagtgggccaagtacatcccggccttctgcgaactccttctggatgaccaggtggcgctgctcagggcccacgccggtgagcatctgctgcttggagccaccaagaggtccatggtgtttaaggacgtgctgctcctaggcaatgactacatcgtccctcggcactgtccagagctagcggagatgagccgtgtgtccatccgcatcctcgatgagctggtcctgcccttccaagagctgcagattgatgacaatgaatatgcctgcctcaaagccatcatcttctttgatccagatgccaaggggctgagtgacccgggcaagatcaagcggctgcggtcacaggtgcaagtgagcctggaggattacatcaacgaccggcagtacgactctcggggccgctttggagagctgctgctgctgttgcccacgctgcagagcatcacctggcagatgatcgaacagatccagttcatcaagctcttcggcatggccaagattgacaacctgctgcaggagatgcttctcggagggtctgccagtgatgcaccccacacccaccaccccctgcaccctcacctgatgcaagaacacatgggcaccaatgtcattgttgctaacacgatgccctctcacctcagcaatggacagatgtgtgagtggccccgacccagggggcaggcagccactcccgagactccacagccatcaccaccaagtggctcgggatctgaatcctacaagctcctgccaggagccatcaccaccatcgtcaagcctccctctgccattccccagccaacgatcaccaagcaagaagccatctag

SEQ ID NO: 18 - protein, 474 aa, Mus musculus
MRLSKTLAGMDMADYSAALDPAYTTLEFENVQVLTMGNDTSPSEGANLNSSNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSQQITSPISGINGDIRAKKIANITDVCESMKEQLLVLVEWAKYIPAFCELLLDDQVALLRAHAGEHLLLGATKRSMVFKDVLLLGNDYIVPRHCPELAEMSRVSIRILDELVLPFQELQIDDNEYACLKAIIFFDPDAKGLSDPGKIKRLRSQVQVSLEDYINDRQYDSRGRFGELLLLLPTLQSITWQMIEQIQFIKLFGMAKIDNLLQEMLLGGSASDAPHTHHPLHPHLMQEHMGTNVIVANTMPSHLSNGQMCEWPRPRGQAATPETPQPSPPSGSGSESYKLLPGAITTIVKPPSAIPQPTITKQEAI

SEQ ID NO: 19 - DNA, 1080 bp, Mus musculus
atggagtcggccgacttctacgaggtggagccgcggcccccgatgagcagtcacctccagagccccccgcacgcgcccagcaacgccgcctttggctttccccggggcgcgggccccgcgccgcccccagccccacctgccgccccggagccgctgggcggcatctgcgagcacgagacgtctatagacatcagcgcctacatcgacccggccgccttcaacgacgagttcctggccgacctcttccagcacagccgacagcaggagaaggccaaggcggcggcgggccccgcgggtggcggcggtgactttgactacccgggagccccggcgggccccggcggcgcggtcatgtccgcgggggcgcacgggccccctcccggctacggctgtgcggcggccggctacctggacggcaggctggagcccctgtacgagcgcgtcggggcgcccgcgctacggccgctggtgatcaaacaagagccccgcgaggaggacgaggcgaagcagctggcgctggccggcctcttcccctaccagccaccgccgccaccgccaccgccgcacccgcacgcgtctcccgcgcacctggccgccccccacttgcagttccagatcgcgcactgcggccagaccaccatgcacctgcagcctggccaccccacaccgccgcccacgcccgtgcccagcccgcacgctgcgcccgccttgggtgctgcgggcctgcctggccccgggagcgcgctcaagggcttggccggtgcgcaccccgacctccgcacgggaggcggcggcggtggcagcggtgccggtgcgggcaaagccaagaagtcggtggacaagaacagcaacgagtaccgggtacggcgggaacgcaacaacatcgcggtgcgcaagagccgagataaagccaaacaacgcaacgtggagacgcaacagaaggtgctggagttgaccagtgacaatgaccgcctgcgcaagcgggtggaacagctgagccgtgaactggacacgctgcggggcatcttccgccagctgcctgagagctccttggtcaaggccatgggcaactgcgcgtga

SEQ ID NO: 20 - protein, 359 aa, Mus musculus
MESADFYEVEPRPPMSSHLQSPPHAPSNAAFGFPRGAGPAPPPAPPAAPEPLGGICEHETSIDISAYIDPAAFNDEFLADLFQHSRQQEKAKAAAGPAGGGGDFDYPGAPAGPGGAVMSAGAHGPPPGYGCAAAGYLDGRLEPLYERVGAPALRPLVIKQEPREEDEAKQLALAGLFPYQPPPPPPPPHPHASPAHLAAPHLQFQIAHCGQTTMHLQPGHPTPPPTPVPSPHAAPALGAAGLPGPGSALKGLAGAHPDLRTGGGGGGSGAGAGKAKKSVDKNSNEYRVRRERNNIAVRKSRDKAKQRNVETQQKVLELTSDNDRLRKRVEQLSRELDTLRGIFRQLPESSLVKAMGNCA

(19) United States
(12) Patent Application Publication
(10) Pub. No.: US 2013/0130297 A1
(43) Pub. Date: May 23, 2013

(54) INDUCTION OF A MATURE HEPATOCYTE PHENOTYPE

(71) Applicant: Michael Ott, Wunstorf (DE)

(72) Inventor: Michael Ott, Wunstorf (DE)

(73) Assignee: MEDIZINISCHE HOCHSCHULE HANNOVER, Hannover (DE)

(21) Appl. No.: 13/697,233

(22) PCT Filed: May 9, 2011
(86) PCT No.: PCT/EP2011/057382

§ 371 Date: Jan. 28, 2013

(30) Foreign Application Priority Data
May 10, 2010 (EP) 10004938.6

(51) Int. Cl. C12N 5/071

(52) U.S. Cl. CPC C12N 5/067
USPC 435/29; 435/455; 435/377

(57) ABSTRACT

The present invention relates to a method for producing cells having a mature hepatocyte phenotype, comprising (a) providing a cell population comprising precursor cells of mature hepatocytes, wherein said cell population is obtained from a subject or derived from a cell line; and (b) introducing into said precursor cells a group of differentiation factors and/or the nucleic acid sequences encoding said differentiation factors, said group consisting of (i) one or more member(s) of the Foxa subfamily, (ii) HNF-4α and (iii) C/EBPα, thereby differentiating said precursor cells into cells having a mature hepatocyte phenotype.

The present invention relates to a method for producing cells having a mature hepatocyte phenotype, comprising (a) providing a cell population comprising precursor cells of mature hepatocytes, wherein said cell population is obtained from a subject or derived from a cell line; and (b) introducing into said precursor cells a group of differentiation factors and/or the nucleic acid sequences encoding said differentiation factors, said group consisting of (i) one or more member(s) of the Foxa subfamily, (ii) HNF-4α and (iii) C/EBPα, thereby differentiating said precursor cells into cells having a mature hepatocyte phenotype.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

In recent years the new concept of programmed cell fate change and differentiation through concerted expression of transcription factors has been emerged. This concept bypasses the state of pluripotency and instead directly induces the desired phenotype by transferring and expressing multiple transcription factors which were shown to change gene expression patterns and to induce cell fate changes in embryonic and adult stem/progenitor cells.

Takahashi and Yamanaka (Takahashi et al., 2006, 2007) were the first to show that the expression of a particular combination of transcription factors can induce a cell fate change and reprogram adult cells into embryonic stem cell like cells. In more detail, Takahashi and Yamanaka set a new paradigm with their experimental approach to reprogram mouse embryonic/adult fibroblasts to ES-like stem cells, referred to as induced pluripotent (iPS) cells, by retroviral transfer of the transcription factors Oct4, Sox2, c-Myc and Klf4. Although the differentiation potential of iPS cells is variable, meanwhile cells from all three germ layers including functional hepatocyte-like cells have been generated with protocols similar to those applied for ES cells. Using a similar approach Vierbuchen (Vierbuchen et al., 2010) transferred a set of transcription factors to directly induce a neuro-phenotype in fibroblasts.

However, to the best knowledge of the inventors so far no study has attempted multiple ectopic transcription factor expression for the induction of a mature hepatic phenotype. Currently various protocols for directed hepatic differentiation of stem cells have been developed in the prior art as alternatives for large scale hepatocyte production. For instance, embryonic stem cells can be grown in unlimited numbers and differentiate into functional hepatocyte-like cells in culture. Maintenance, growth and differentiation, however, are time consuming and expensive. Differentiation of cells takes up to 40 days and result in a cell population of cells with variable degrees of differentiation. Further, embryonic stem cells, for now, are not considered for cell therapy applications due to risks for teratoma formation and ethical concerns. In recent years, evidence has been provided that also adult cells from various sources (bone marrow/adipose tissue/placenta/umbilical cord) could occasionally overcome lineage borders and differentiate into hepatic lineage cells upon coordinated in vitro stimulation (Sgodda M, et al. (2007), Yamazaki S et al., (2003), Kang X Q, et al. 2005. Ong S Y et al (2006)). Despite remarkable progress, the resulting cells often fail to achieve complete function sufficient for regenerative therapy and toxicology assays or other pharmacological assays, remaining only, “hepatocyte-like”.

Therefore, mature hepatocytes are still frequently needed for in vitro toxicology or pharmaceutical assays and also potentially for cell therapies in patients with acute and metabolic liver diseases. However, liver tissues for hepatocyte isolation are rarely available. A further drawback is that hepatocytes show limited proliferation potential and dedifferentiate over time in culture. Thus, the isolation of high quality hepatocytes from organs and tissues for toxicological studies and clinical therapies remains a challenge and methods to obtain alternative cellular resources are urgently needed.

This need is addressed by the provision of the embodiments characterized in the claims.

Accordingly, in a first embodiment the invention relates to a method for producing cells having a mature hepatocyte phenotype, comprising (a) providing a cell population comprising precursor cells of mature hepatocytes, wherein said cell population is obtained from a subject or derived from a cell line; and (b) introducing into said precursor cells a group of differentiation factors and/or the nucleic acid sequences encoding said differentiation factors, said group consisting of (i) one or more member(s) of the Foxa subfamily, (ii) HNF-4α and (iii) C/EBPα, thereby differentiating said precursor cells into cells having a mature hepatocyte phenotype.

In accordance with the invention the term “precursor cell” refers to a cell having the capability to differentiate into a mature cell. Thus, a precursor cell specifies a cell which is partially or fully undifferentiated. With regard to the present invention, the precursor cell is a partially differentiated cell or a fully undifferentiated cell and has the capability to differentiate into a cell having a mature hepatocyte phenotype.

The term “mature hepatocyte” as used herein specifies the differentiated parenchymal cell of the liver. Hepatocytes make up 70-80% of the liver's cytoplasmic mass and are the predominant cell type in the liver. Hepatocytes are cells, for example characterized by the secretion of serum albumin, fibrinogen, and the prothrombin group of clotting factors (except for Factor 3 and 4). Moreover, hepatocytes are the main sites for the synthesis of lipoproteins, ceruloplasmin, transferrin, complement, and glycoproteins, nutrient metabolism and xenobiotic transformation.

The term “subject” as used herein means a vertebrate, preferably a mammal, more preferably any one of cat, dog, horse, cattle, swine, goat, sheep, mouse, rat, monkey, ape and human, and most preferably a human.

The term “differentiation factor” in accordance with the invention specifies any compound which when contacted with one or more precursor cell(s) leads to a more differentiated cellular stage, either when used alone or in combination with other differentiation factor(s). The term “transcription factor” also used herein specifies a specific subclass of the umbrella term “differentiation factors”. To explain further, a transcription factor specifies a protein that binds to specific DNA sequences and thereby controls the transcription of genetic information from DNA to mRNA. In turn, the protein encoded by the mRNA leads to a more differentiated cellular stage. Thus, a transcription factor falls within the definition of a differentiation factor.

The term “introducing” as used herein specifies the process of bringing a protein and/or nucleic acids sequence into a living cell, an in particular also into the cell nucleus, preferably by introducing means as defined herein below. In accordance with the invention, protein to be introduced is (i) one or more member(s) of the Foxa subfamily, (ii) HNF-4α and (iii) C/EBPα and the nucleic acid sequences is the nucleic acid sequence encoding said differentiation factors

The term “Foxa family” specifies the subfamily a of Forkhead box transcription factors. The Foxa family comprises Foxa1 (human DNA/protein SEQ ID NOs: 1 and 2; mouse DNA/protein SEQ ID NOs. 11 and 12), Foxa2 (human DNA/protein SEQ ID NOs: 3 and 4; mouse DNA/protein SEQ ID NOs. 13 and 14) and Foxa3 (human DNA/protein SEQ ID NOs: 5 and 6; mouse DNA/protein SEQ ID NOs. 15 and 16) (Kaestner et al. (2000), Genes Dev. 14(2):142-146). Also encompassed are fragments of Foxa as long as these fragments retain their differentiation effect on the precursor cells used in accordance with the invention.

The term “HNF-4α” specifies a nuclear receptor of the hepatocyte nuclear factor 4 subfamily that binds to DNA (human DNA/protein SEQ ID NOs: 7 and 8; mouse DNA/protein SEQ ID NOs. 17 and 18) (Chartier et al. (1994). Gene 147 (2): 269-72 and Wisely et al. (2002). Structure 10 (9): 1225-34.). Also encompassed are fragments of HNF-4α as long as these fragments retain their differentiation effect on the precursor cells used in accordance with the invention.

The term “C/EBPα” refers to the a member of the CCAAT-enhancer-binding proteins (or C/EBPs) which are a family of transcription factors (human DNA/protein SEQ ID NOs: 9 and 10; mouse DNA/protein SEQ ID NOs. 19 and 20) (Ramji et al. (2002), Biochem. J. 365:561-575 and Kovács et al (2003), J Biol Chem., 278(38):36959-65). Also encompassed are fragments of C/EBPα as long as these fragments retain their differentiation effect on the precursor cells used in accordance with the invention.

The term “nucleic acid molecule” is defined herein below.

The method of the invention, in particular step (b), is carried out under suitable cell culture conditions. It is of note that cell culture conditions for a cell population comprising precursor cells of mature hepatocytes obtained from a subject or derived from a cell line are well known in the art (e.g. Cooper G M (2000). “Tools of Cell Biology”, ISBN 0-87893-106-6; K. Turksen, ed., Humana Press, 2004, “Adult stem cells” ISBN-10 1-58829152-9, J. Masters, ed., Oxford University Press, 2000, “Animal cell culture”. ISBN-10 0-19-963796-2). Suitable culture conditions are for example shown in more detail in the Examples of the invention (e.g. the liver differentiation media LD1 and LD 2).

The differentiation factors consisting of (i) one or more member(s) of the Foxa subfamily, (ii) HNF-4α and (iii) C/EBPα are either introduced simultaneously or sequentially in the method of the invention. In this regard it is preferred that the factor according to (i) is introduced before using, e.g. adding factors according to (ii) and (iii), and even more preferred that first the factor according to (i), second the factor according to (ii) and third the factor according to (iii) is introduced, e.g. added. In this regard the term “introduced” also embraces the term “expressed”.

Induction and maintenance of hepatocyte differentiation and control of liver-specific gene expression is attributed to a large extent to hepatocyte nuclear factors (HNFs) (Kyrmizi et al., 2006, Lemaigre F P, 2009). This class of proteins includes five families of transcriptional regulators: HNF1, Foxa (formerly HNF3), C/EBP, HNF4, and HNF6. While none of these factors is exclusively expressed in the liver, the combinatorial actions of tissue-specific transcription factors collaborate to achieve the stringency and dynamic regulation of gene expression required for the proper development and function of the organ.

The vertebrate Foxa subfamily is closely related to the Drosophila protein FORK HEAD and comprises Foxa1, Foxa2 and Foxa3. Foxa proteins function as transcriptions factors, as defined by the presence of sequence-specific DNA-binding activity and the ability to regulate transcription of target genes. Foxa proteins are required for normal development of endoderm derived organs such as the liver, pancreas, lungs and prostate. Recent studies have provided evidence for the hypothesis that Foxa proteins act as “competence factors” and facilitate binding and transcriptional activity of other transcription factors in endoderm tissues and cells. Moreover it has been shown that Foxa2 is required for normal liver homeostasis also in the adult liver, as >43% of genes expressed in the liver were also associated with Foxa2 binding (Wederell E 2008).

The hepatic nuclear receptor HNF4α is a key regulator of both hepatocyte differentiation during embryonic development and maintenance of a differentiated phenotype in the adult (Zaret K and Grompe M, 2008 Hayhurst et al., 2008, Parviz F., et al. 2003, Li J et al. 2000). Recent studies using mouse embryos, in which the Hnf4 gene is ablated specifically from fetal hepatocytes, have shown that Hnf4α is essential for the generation of a hepatic epithelium. Furthermore, in the same study, it was demonstrated that HNF4α could induce a mesenchymal to epithelial transition when expressed in naive NIH 3T3 fibroblasts.

Similarly, expression of C/EBPα is critically important for maintaining the differentiated state of hepatocytes in vivo. It is downregulated in rat liver nodules and HCCs and forced expression of C/EBPα was found to impair proliferation and suppress tumorigenicity (Nerlov C., 2007, Tseng H H et. al, 2009). In adult mice, conditional knockout experiments have shown that C/EBPa plays an important role in hepatic glucose, nitrogen, bile acid and iron metabolism all of which represent highly differentiated hepatocyte functions (Tan et al. (2007)).

The inventors have surprisingly found that only three distinct transcription factors, namely (i) one or more member(s) of the Foxa subfamily, (ii) HNF-4α and (iii) C/EBPα proteins selected from the members of the five families of transcriptional regulators for hepatocyte differentiation, namely HNF1, Foxa, C/EBP, HNF4, and HNF6 are essential for appropriate differentiation of precursor cells into cells having a hepatocyte phenotype. In more detail, in the examples provided herein the ability to induce a mature hepatocyte phenotype, for example in clonally expanded adult liver derived progenitor cells (ALDP's) by sequential lentiviral over-expression of this set of liver enriched transcription factors, namely Foxa2, HNF4α and C/EBPα is shown.

The method provided in accordance with the invention allow for large scale hepatocyte production. Moreover, the method of the invention is, to the best knowledge of the inventors, a more rapid (7 days as shown in the appended examples) and efficient differentiation approach as compared to the methods known in the art.

As known in the art, induction and maturation of a hepatocyte phenotype in the embryonic liver is governed by the orderly and hierarchical expression of liver enriched transcription factors. As explained in more detail herein above, for example the hepatic nuclear factors and CCAT/Enhancer binding proteins play key roles in the developing liver as well as in the regulation of liver specific gene expression in the adult organ. However, whereas the inventors do not wish to be bound by any theory, it is hypothesized that over-expression of members of these transcription factor families could induce and maintain a mature hepatic phenotype in a liver progenitor cell line, it was not known which transcription factors or more general which differentiation factors are essential and sufficient to induce and maintain a mature hepatic phenotype. As shown in the examples, the three transcription factors, namely Fox2a, HNF-4α and C/EBPα expressed in ALDP cells are essential. These factors were selected based on known functions in liver development and most importantly based on the following criteria set and investigated by the inventors: (1)>10 fold up-regulation during in mouse embryonic liver compared to whole embryo tissue, (2)>10 fold downregulation in cultured hepatocytes over a culture period of six days and (3)>3 fold induction in embryonic stem cells throughout the hepatic differentiation process. Applying these innovative criteria led to the identification of Fox2a, HNF-4α and C/EBPα and the unexpected finding that the morphological and metabolic phenotype of triple transduced ALDP cells surprisingly closely resemble mature mouse hepatocytes.

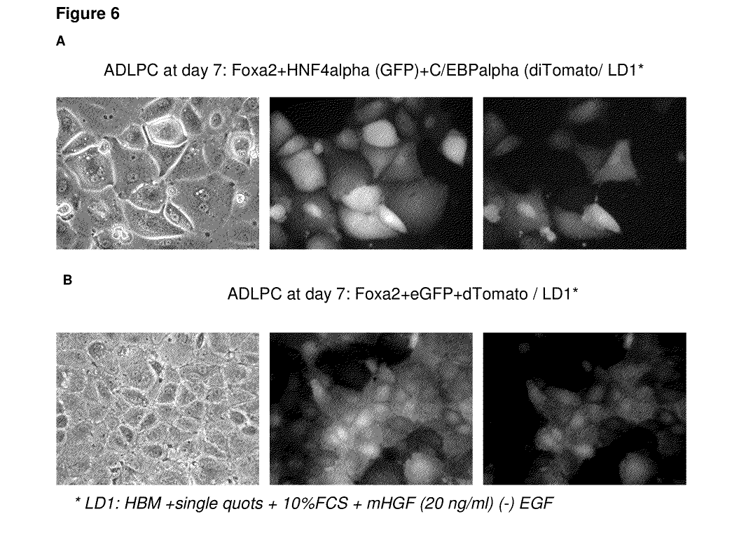

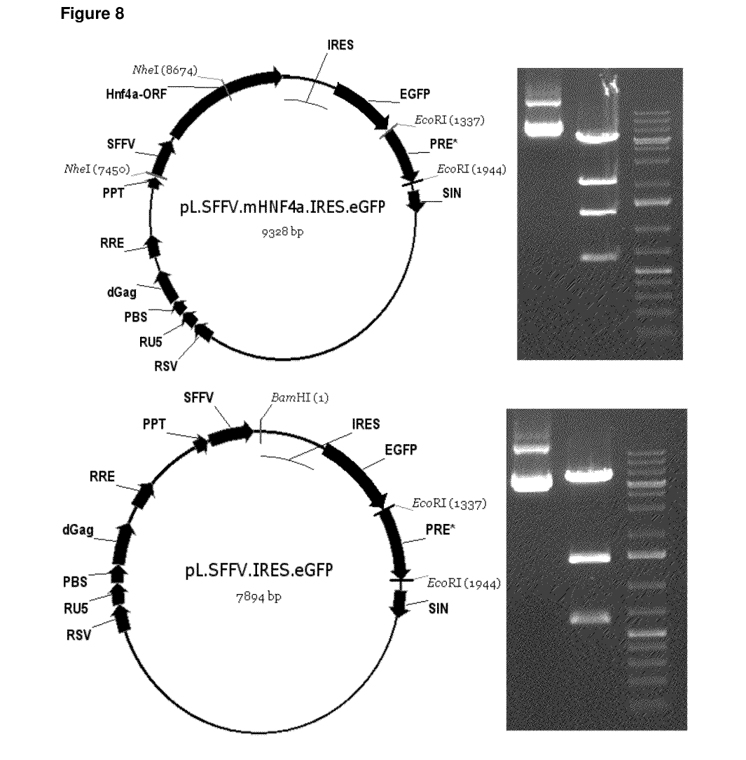

The mature mouse hepatocytes phenotype is evidenced by the fact that cultures treated with all three transcription factors contained significantly more cells with two or more nuclei, which is a hallmark of adult mouse hepatocytes. Moreover, an incremental increase in albumin secretion in single, double and triple transduced cells is observed. To the best knowledge of the inventors, it is for the first time reported that albumin secretion of differentiated hepatocyte-like cells is identical to adult hepatocytes (cf. FIG. 8). PAS positive staining, which suggests glycogen storage ability, was also comparable to adult hepatocytes (cf. FIG. 6). As previously reported, Foxa2 overexpression in mesenchymal stem cells generated only marginal PAS positivity when compared to HUH7 controls (Ishii et al. (2008). Ureagenesis was also maximally induced in triple transduced cells, however at a much lower level compared to day 1 cultured hepatocytes. Although the expression of carbamoylphosphate synthase-1 (CPS-1), a rate-limiting enzyme in urea synthesis, was reported to be strongly decreased in the absence of C/EBPα (Inoue et al. (2004)), the over-expression of this transcription factor in the experimental setting does not seem to be enough to bring ureagenesis in the hepatocyte range. Over-expression of C/EBPα even induces toxic effects in ALDP cells and fibroblast cultures in the absence of HNF4α overexpression and this effect seems to be dose and time-dependent. This finding is consistent with previous reports that ectopic expression of C/EBPα gene could induce hepatic stellate cells apoptosis in a dose- and time-dependent manner as shown by Annexin V/PI staining, caspase-3 activation assay, and flow cytometry (Wang et al. (2009)). It further suggests that optimal effects of CEBPα which lead to terminal hepatic differentiation require fine tuning and can be achieved only in a predifferentiated hepatocyte-like state.

Although also prior art studies on hepatocyte differentiation report positive functional testing for hepatocyte characteristics, the positive controls used were not cultured mature adult hepatocytes. In other words, the efficiency of hepatic differentiation is difficult to appreciate in comparison with other cells than mature adult hepatocytes and most likely did not reach the level of mature hepatocytes as shown herein for the cells differentiated according to the method of the invention.

In more detail, in the prior art it is has only been shown that mouse ES cells were able to differentiate into hepatocyte-like cells with liver specific metabolic functions when Foxa2 transfected cells were grown in α-MEM supplemented with FGF2, nicotinamide dexamethasone, L-ascorbic-2-phosphate and 10% FBS in a 3 dimensional culture system to form spheroids (Ishikaza et al (2002)). In a recent study, stage specific forced expression of homeobox gene Hex in embryoid bodies from wild type ESCs led to the up-regulation of albumin and Afp expression and secretion of albumin and transferrin. Microarray analysis showed that Hex regulated the expression of a broad spectrum of hepatocyte-related genes, including fibrinogens, apolipoproteins, and cytochromes. The effects were restricted to c-kit(+) endoderm-enriched EB-derived populations, suggesting that Hex functions at the level of hepatic specification of endoderm (Kubo et al. (2010)). Ishii et al. loc. lit. have established a tetracycline-regulated expression system for Foxa2 in UE7T-13 BM-MSCs showing that expression of Foxa2 significantly enhanced expression of albumin, alphafetoprotein (AFP), tyrosine amino transferase (TAT) and epithelial cell adhesion molecule (EpCAM) genes. The differentiated cells showed hepatocyte-specific functions including glycogen production and) urea secretion, however only when compared to the cell lines HUH-7 or HepG2 controls.

In conclusion, it is shown in accordance with the present invention that expression of Foxa2, HNF4α and C/EBPα induces a mature hepatocyte-like phenotype, for example in an expandable liver derived progenitor cell line leading to better results with regard to the analogy to mature hepatocytes as compared to conventional differentiation protocols. The excellent expansion capability of the ADLPCs used and the findings with regard to their capacity to differentiate to cells having a mature hepatocyte phenotype when applying the method of the invention makes them attractive hepatocyte precursor sources to be used for drug testing studies and as a basis for developing bioartificial liver support devices.

Accordingly the invention furthermore relates to a bioartificial liver support comprising the cells obtained by the method of the invention.

The term “bioartificial liver support” as used herein refers to a compound which may be used in the treatment of a liver disease as defined herein below.

In accordance with a preferred embodiment, the method of the invention further comprises: (c) isolating cells having a mature hepatocyte phenotype from the cell population obtained in step (b) above.

Methods for isolating cells are well known in the state of the art and comprise FACS (fluorescence activated cell sorting), sucrose gradient centrifugation, (laser) microdissection, single cell dilution, and Magnetic Labeled Bead Cell Separation. FACS is a method for sorting a heterogeneous mixture of biological cells into two or more containers, one cell at a time, based upon the specific light scattering and fluorescent characteristics of each cell. For this purpose a cell specific marker. e.g. a hepatocyte-specific cell-surface marker (for example the a hepatocyte-specific cell-surface marker cytokeratin 8 or 18) may be labeled with a fluorescent dye like FLAG, GFP, YFP, RFP, dTomato, cherry, Cy3, Cy 5, Cy 5.5, Cy 7, AMCA, Tamra, Texas Red, rhodamine, Alexa fluors, FITC or TRITC. Sucrose gradient centrifugation is a centrifugation technique which separates compounds according to their density along a sucrose density gradient which is typically created overlaying lower concentrations of sucrose on higher concentrations in a centrifuge tube. Single cell dilution is a technique of diluting cells, preferably in culture medium until a concentration is reached that allows to separate single cell in a separated vial. Magnetic Labeled Bead Cell Separation is a commercial system available from Miltenyi Biotec (MACS), Bergisch Gladbach, Germany.

In a more preferred embodiment the method of the invention the introduction into the precursor cells is effected by transforming precursor cells of the cell population of (a) with one or more nucleic acids molecule(s) comprising the nucleic acid sequences encoding (i) one or more member(s) of the Foxa subfamily, (ii) HNF-4α and (iii) C/EBPα in an expressible form.

The term “nucleic acid molecule”, in accordance with the present invention, includes DNA, such as cDNA or genomic DNA, and RNA. It is understood that the term “RNA” as used herein comprises all forms of RNA including mRNA. The term “nucleic acid molecule” is interchangeably used in accordance with the invention with the term “polynucleotide”.

The nucleic acid molecule(s) may also comprise regulatory regions or other untranslated regions. In a further embodiment, said nucleic acid molecule may comprise heterologous nucleic acid which may encode heterologous proteinaceous material thus giving rise, e.g., to fusion proteins.

The present invention thus relates to a the nucleic acid molecule(s) of the invention encoding a fusion protein. For example, the nucleic acid molecule of the invention can be fused in frame to a detectable marker such as purification marker or a direct or indirect fluorescence marker. Purification marker comprise one or more of the following tags: His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

Methods for transformation of cells are well established in the state of the art and include but are not limited to viral transformation (e.g. adenoviral, adeno-associated, retroviral, lentiviral transfection), transposon transformation (e.g. retrotransposons, DNA-transposons, retroviruses as transposable elements, and in particular Tc1/mariner-class transposons like piggyBac and Sleeping Beauty), lipofection, microinjection, electroporation, impalefection, gene gun, magnetofectin, sono-poration, optical transfection (e.g. by a laser), and chemical-based transfection.

In an even more preferred embodiment of the method of the invention the one or more nucleic acids molecule(s) are one or more vector(s).

Accordingly, the present invention also relates to a vector containing the nucleic acid molecule(s) of the present invention. Preferably, the vector is a plasmid, cosmid, virus, bacteriophage or another vector used e.g. conventionally in genetic engineering.

The nucleic acid molecule may be inserted into several commercially available vectors. The nucleic acid molecule(s) referred to above may also be inserted into vectors such that a translational fusion with another polynucleotide is generated. The other polynucleotide may encode a protein which may e.g. increase the solubility, correct protein folding, facilitate the purification of the cells of the invention and/or further differentiation factors enhancing differentiation and/or maintenance of the mature hepatocyte phenotype. For vector modification techniques, see Sambrook and Russel (2001), loc. cit. Generally, vectors can contain one or more origin of replication (ori) and inheritance systems for cloning or expression, one or more markers for selection in the host, e.g. dihydrofolate reductase, G418, neomycin, ampicillin, hygromycin, or kanamycin, and one or more expression cassettes. Suitable origins of replication (ori) include, for example, the Col E1, the SV40 viral and the M 13 origins of replication.

The coding sequences inserted in the vector can e.g. be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression in prokaryotes or eukaryotic cells are well known to those skilled in the art. These elements comprise regulatory sequences ensuring the initiation of transcription (e.g., translation initiation codon, promoters, such as naturally-associated or heterologous promoters and/or insulators), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers.

An expression vector according to this invention is capable of directing the replication, and the expression, of the polynucleotide and encoded differentiation factor(s) of this invention. Suitable expression vectors which comprise the described regulatory elements are known in the art. The nucleic acid molecules as described herein above may be designed for direct introduction, phage vectors or viral vectors (e.g. adenoviral, retroviral) into a cell.

A typical mammalian expression vector contains the promoter element, which mediates the initiation of transcription of mRNA, the protein coding sequence, and signals required for the termination of transcription and polyadenylation of the transcript and moreover, elements such as origin of replication, drug resistance gene, regulators (as part of an inducible promoter) may also be included. Highly efficient transcription can be achieved with the early and late promoters from SV40, the long terminal repeats (LTRs) from retroviruses, e.g., RSV, HTLVI, HIVI, and the early promoter of the cytomegalovirus (CMV). Alternatively, the recombinant polypeptide can be expressed in stable cell lines that contain the gene construct integrated into a chromosome. The co-transfection with a selectable marker such as dhfr, gpt, neomycin, hygromycin allows the identification and isolation of the transfected cells. The transfected nucleic acid can also be amplified to express large amounts of the encoded polypeptide.

In an even more preferred embodiment of the method of the invention the one or more vector(s) are one or more lentiviral vector(s).

Examples of lentiviruses providing elements of a lentiviral vector are HIV, SIV, FIV, Puma lentivirus, EIA, Bovine immunodeficiency virus, Caprine arthritis encephalitis virus, or Visna/maedi virus. Preferably used in accordance with the invention is a 3^{rd} generation lentiviral expression plasmid containing a SFFV promoter driven IRES-Puro (Foxa2), IRES-dTomato (C/EBPα) or IRES-eGFP (HNF4α) expression cassette. More preferably, one or more vectors as shown in FIGS. 8 to 10 are used. Lentiviruses are particularly useful in the method for the invention because they integrate into the genome and thus allow for non-transient gene expression. Moreover, studies have shown that lentivirus vectors have a lower tendency to integrate in places that potentially cause cancer than gamma-retroviral vectors which also integrates into the genome (Cattoglio et al. (2007), Blood, 110(6) 770-1778). Thus they have a lower tendency of abnormal cell growth upon integration.

In accordance with a more preferred embodiment of the method of the invention the nuclei acid sequences encode (i) the one or more member(s) of the Foxa subfamily, (ii) HNF-4α and (iii) C/EBPα in an inducible expressible form.

Systems for inducible expression are widely used in the state of the art and include but are not limited to the Tet-on system, Tet-off system, lac operator/expressor system and the commercial systems Q-mate™, Rheo Swith®, Cumate® or TREX™. As known in the art inducible expression systems allow to control the expression of a gene over time, the order of the genes expressed (provided that different expression systems, e.g. a Tet-on system and Tet-off system are used in parallel or subsequently for the expression of different genes) and also the amount of expressed mRNA from the vector.

In a further more preferred embodiment the method of the invention the introduction into the precursor cells is effected by microinjection, electroporation, lipofection and/or protein transduction of (i) one or more member(s) of the Foxa subfamily, (ii) HNF-4α and (iii) C/EBPα into said precursor cells.

This Methods for introducing a protein (or peptide) directly into a cell are well known in the state of the art and comprise but are not limited to microinjection, electroporation, lipofection (using liposomes) and protein transduction. In this regard, the proteins to be introduced may either be isolated form their natural environment or recombinantly produced. These methods are capable to bypass or further support the step of transforming a cell as described herein above.

A liposome used for lipofection is a small vesicle, composed of the same material as a cell membrane (i.e., normally a lipid bilayer e.g. out of phospholipids), which can be filled with one more protein(s) (e.g. Torchilin V P. (2006), Adv Drug Deliv Rev., 58(14):1532-55). To deliver a protein into a cell, the lipid bilayer of the liposome can fuse with the lipid bilayer of the cell membrane, thereby delivering the contained protein into the cell. It is preferred that the liposomes used in accordance with invention are composed of cationic lipids which. The cationic liposome strategy has been applied successfully to protein delivery (Zelphati et al. (2001). J. Biol. Chem. 276, 35103-35110). As known in the art, the exact composition and/or mixture of cationic lipids used can be altered, depending upon the protein(s) of interest and the cell type used (Felgner et al. (1994). J. Biol. Chem. 269, 2550-2561).

Protein transduction specifies the internalisation of proteins into the cell, from the external environment (Ford et al (2001), Gene Therapy, 8:1-4). This method relies on the inherent property of a small number of proteins and peptides (preferably 10 to 16 amino acids long) being able to penetrate the cell membrane. The transducing property of these molecules can be conferred upon proteins which are expressed as fusions with them and thus offers, for example, an alternative to gene therapy for the delivery of therapeutic proteins into target cells. Commonly used proteins or peptides being able to penetrate the cell membrane are, for example; the antennapedia peptide, the herpes simplex virus VP22 protein, HIV TAT protein transduction domain, peptides derived from neurotransmitters or hormones, or a 9×Arg-tag.

Microinjection and electroporation are well known in the art and the skilled person knows how to perform these methods. Microinjection refers to the process of using a glass micropipette to introduce substances at a microscopic or borderline macroscopic level into a single living cell. Electroporation is a significant increase in the electrical conductivity and permeability of the cell plasma membrane caused by an externally applied electrical field. By increasing permeability protein (or peptides or nucleic acid sequences) can be introduced into the living cell.

In accordance with a preferred embodiment of the method of the invention the member of the Foxa subfamily is Foxa2.

The member Fox2a of the Foxa family has been used in the examples of the invention provided herein below. Thus, the use of Fox2a is preferred.

In another preferred embodiment of the method of the invention the precursor cells of mature hepatocytes are embryonic stem cells, adult stem cells, or iPS cells.

Stem cells like embryonic stem cells, adult stem cells, or iPS cells are characterized by their ability to renew themselves through mitotic cell division and differentiate into a diverse range of specialized cell types, including hepatocytes. Accordingly, embryonic stem cells, adult stem cells, or iPS cells are particularly suitable precursor cells in accordance with the invention.

The embryonic stem cells as far as being human embryonic stem cells were not established by the inventors by a process involving the step of disrupting a human embryo. In other words, the method of the invention is practised without the need of disrupting a human embryo, by for example using human embryonic stem cell lines established and available at the time the invention was made. Such cells are inter alia described in Thomson et. al (1998), “Blastocysts Embryonic Stem Cell Lines Derived from Human”, Science 282 (1145): 1145-1147, or Thomson et al. (1998), “Embryonic stem cell lines derived from human blastocysts”, Science 282 (5391): 1145-7. Specific examples are the cell lines CHB-1, CHB-2, or CHB-3.

In an alternative preferred embodiment of the method of the invention the precursor cells of mature hepatocytes are (a) primitive epithelial progenitor cells obtained from liver suspension, or (b) adult liver derived progenitor cells.

Primitive epithelial progenitor cells obtained from liver suspension, or adult liver derived progenitor cells are precursor cells of mature hepatocytes which already have partly differentiated into the lineage of mature hepatocytes but however—in contrast to fully differentiated mature hepatocytes—retained the capability to grow in vitro. Due to this advantage, they are particularly preferred precursor cells in accordance with the invention. Adult liver derived progenitor cells we also employed in the examples provided by the invention.

Methods to obtain primitive epithelial progenitor cells obtained from liver suspension, or adult liver derived progenitor cells are well established in the state of the art and described, for instance in the examples provided herein.

In more detail, the adult liver derived progenitor cells (ALDPs) can be obtained from adult liver cell suspension cultures in the presence of growth factor supplemented medium and were described as bipotent adult mouse liver (BAML) cells by others. Whether they exist in the liver or derive from hepatocytes or other cell types in the presence of growth factors in culture, is currently not known. The cells are expandable to huge numbers and theoretically can be generated even from a patient's liver biopsy. Further, abnormal cell growth, in particular the formation of a tumour from these cells was not reported in the literature. This makes these cells attractive for cell therapy applications. Proliferation capacity, expression of endoderm markers (Sox17, GATA4), liver enriched transcription factors, E- and N-Cadherins as well as cytokeratins 7, 8, 18, 19 and hepatic and biliary differentiation potential qualifies the cells as progenitors of mature liver cells. To the best knowledge of the inventors, so far attempts to differentiate ALPDs in vitro towards mature hepatocytes, by conventional differentiation factors, has resulted in only marginal induction of hepatic gene expression and hepatocyte functions. As shown in the examples provided herein, ALDPs can effectively be differentiated into cells having a mature hepatocyte phenotype (characterized by gene expression of mature hepatocytes and mature hepatocyte functions) by applying the method of the invention.

Using liver progenitor phenotype cells like ALDP cells in the method of the invention most likely facilitates the hepatic differentiation process induced by the transcription factors. The method of the invention has also been applied to NIH 3T3 and mouse embryonic fibroblasts as precursor cells. Although, the efficacy of hepatic differentiation was less than observed for the ALDP cells, however also these cells could be differentiated into hepatocyte like cells. For example, a dramatic morphological change to an epitheliod phenotype was noted. Moreover albumin expression and expression of several other liver genes could be induced in triple transduced fibroblasts. Although most of the parameters tested did not reach the levels of mature hepatocytes, this shows that the methods described herein can also be applied to other cell types (e.g. fibroblasts, mesenchymal cells or virtually any suitable precursor cell type) or cell lines than ALDP cells which are preferably used.

In a preferred embodiment of the method of the invention the obtained cells are free of pathogens.

The term “pathogen” in accordance with the invention refers to a biological agent that causes or contributes to a disease in the host. Examples of pathogens comprise viral pathogens, fungal pathogens, helminthic pathogens, prionic pathogens, protistic pathogens and bacterial pathogens.

In a preferred embodiment of the method of the invention the cells having a mature hepatocyte phenotype are characterized by the expression of albumin, AAT, TAT, TDO, G6P, Cyp3A7, Cyp2A5, PXR, Apo44, ApoC3 and ApoA1.

The expression of the genes albumin, AAT, TAT, TDO, G6P, Cyp3A7, Cyp2A5, PXR, Apo44, ApoC3 and ApoA1 characterize the known gene expression profile of mature hepatocytes (e.g. Li et al. (2010) Differentiation. 2010 Apr. 26. [Epub ahead of print]. Castro et al. (2005) Journal of Hepatology, 42(6):897-906; or Wu et al. (2002) The FASEB Journal; 16:1665-1667).

In a further preferred embodiment of the method of the invention the cells having a mature hepatocyte phenotype are characterized by the capability to store glycogen intracellularly and the capability to generate urea.

The intracellular storage of glycogen and the capability to generate urea inter alia characterize mature hepatocytes (e.g., Michalopoulos et al. (2002), Am J Pathol. 2001 November; 159(5): 1877-1887; Horslen et al (2003), PEDIATRICS, 111; 1262-1267, or Baquet et al. (1990). Journal of Biological Chemistry, 265, 955-959.).

In a further preferred embodiment of the method of the invention the cells having a mature hepatocyte phenotype are characterized by comprising cells with dispersed chromatin and/or two or more cell nuclei.

Dispersed chromatin and/or two or more cell nuclei are phenotypic characteristics of mature hepatocytes, well known and described in the art (e.g., Michalopoulos et al. (2002), Am J Pathol. 2001 November; 159(5): 1877-1887.).

In a further preferred embodiment of the invention the method of differentiation in step (b) is carried out in the presence of one or more additional differentiation factors selected from the group consisting of histone deacetylase inhibitors.

Although the cells carrying with one or more member(s) of the Foxa subfamily, HNF-4α and C/EBPα exhibit gene expression and protein secretion levels compatible with a mature hepatic phenotype, more complex metabolic functions of hepatocytes such as ureagenesis may still be-optimized. The over-expression of additional transcription factors and three dimensional cultures on scaffolds may further improve the maturation of these cells. In this regard, histone deacetylase inhibitors, which have been shown to improve hepatic differentiation in embryonic and stem cells appear to be suitable for achieving this purpose, because they are known to have role in hepatocytes differentiation (e.g. Yamashita et al. (2002), Cancer Cell Biology 103(5):572-576 or Armeanu et al. (2005), Journal of Hepatology, 42(2):210-217).

According to an embodiment of the invention the cells obtained by the method according to the invention are used for treating or preventing a liver disease.

Accordingly, also described herein is a method of treating or preventing a liver disease comprising administering an pharmaceutically effective amount of cells obtained by the method according to the invention to a subject in need thereof.

Moreover, described herein is a method of treating or preventing a liver disease in a subject in need thereof comprising (a) isolating a cell population comprising precursor cells of mature hepatocytes, wherein said cell population is isolated from said subject; (b) differentiating precursor cells of the cell population into cells having a mature hepatocyte phenotype by introducing into said precursor cells a group of differentiation factors consisting of (i) one or more member(s) of the Foxa subfamily, (ii) HNF-4α and (iii) C/EBPα; and (c) administering an pharmaceutically effective amount of differentiated cells obtained in (c) to said subject.

As shown in the examples of the application, the cells obtained by the method of the invention have the phenotype of mature hepatocytes, however without being identical to mature hepatocytes. Thus, it can be expected that these cells can substitute or partly substitute for mature hepatocytes in vivo, in particular when used for the treatment or inhibition of a liver disease as defined herein.

The term “liver disease” as used herein refers to any pathological condition or injury involving the loss of hepatocytes, or the dysfunction/loss of function of hepatocytes. Thus, the cells obtained by the method of the invention are capable to supplement or partially supplement for mature hepatocytes and/or the function of mature hepatocytes. Liver diseases comprise but are not limited to liver injury, liver failure (i.e. a dysfunction of the liver to perform its normal synthetic and metabolic function as part of normal physiology and comprises acute liver failure and chronic liver failure), hepatitis, cirrhosis, cancer of the liver, glycogen storage disease type II, Gilbert's syndrome, Budd-Chiari syndrome, primary biliary cirrhosis, primary sclerosing cholangitis. Wilson's disease, hepatic encephalopathy, haemochromatosis, urea cycle defects, and non-alcoholic fatty liver disease.

In another embodiment the invention relates to a method for identifying a compound having an pharmacological, cytotoxic, proliferative, transforming or differentiating effect on cells having a mature hepatocyte phenotype obtained by the method described herein, comprising: (a) contacting said cells having a mature hepatocyte phenotype with a test compound; and (e) determining whether the test compound has one or more of said effects on said cells having a mature hepatocyte phenotype.

The cells having mature hepatocyte phenotype obtained by the method described herein are suitable for pharmacological or scientific research and may replace testing of cells directly obtained from liver and/or animal experiments. In this regard compounds may be tested with regard to their effect to cells having the mature hepatocyte phenotype which has been obtained by the method of the invention. Such effects comprise pharmacological, cytotoxic, proliferative, transforming or differentiating effect. Such effects may be for example monitored by measuring cell proliferation, apoptosis, cell morphology, cell migration, gene expression, cellular protein, or metabolic processes.

The figures show:

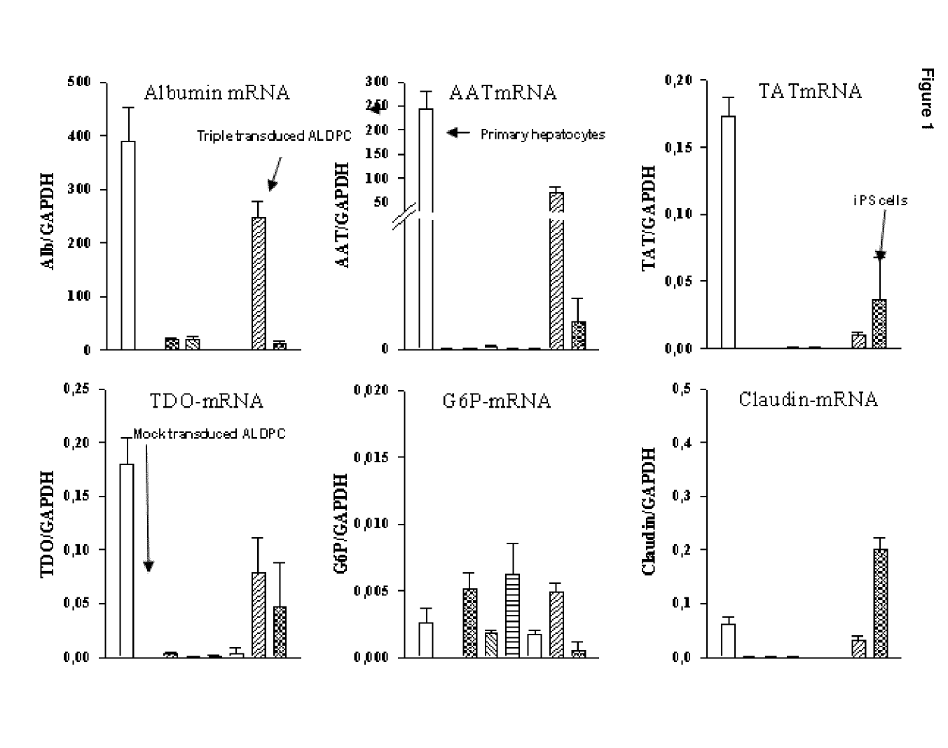

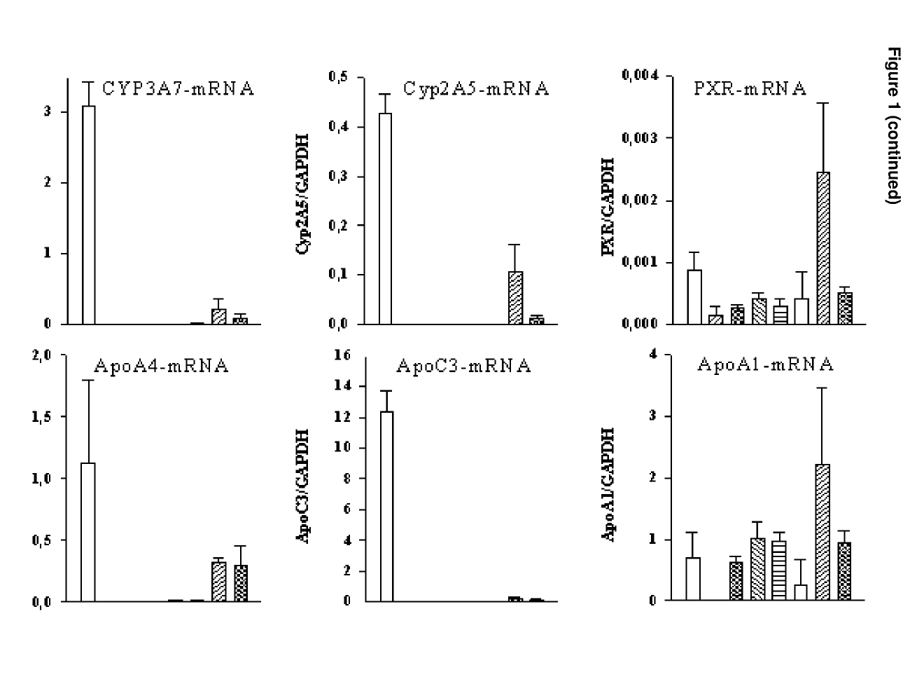

FIG. 1: mRNA expression levels relative to GAPDH mRNA expression of the genes albumin, AAT, TAT, TDO, G6P, Claudin1, Cyp3A7, Cyp2A5, PXR, ApoA4, ApoC3, ApoA1 in primary mouse hepatocytes (14 h cultured, column 1). ADLPC in basic growth medium condition (column 2), in hepatic differentiation medium LD1, adherent culture (column 3), in differentiation medium LD2, adherent culture (column 4), in differentiation medium LD 1, non-adherent culture (column 5), in differentiation medium LD 2, non-adherent culture (column 6), in LD1 after triple transduction with transcription factors (FoxA2, HNF4, C/EBPα), adherent culture (column 7) and in iPS cells differentiated towards the hepatic lineage (column 8).

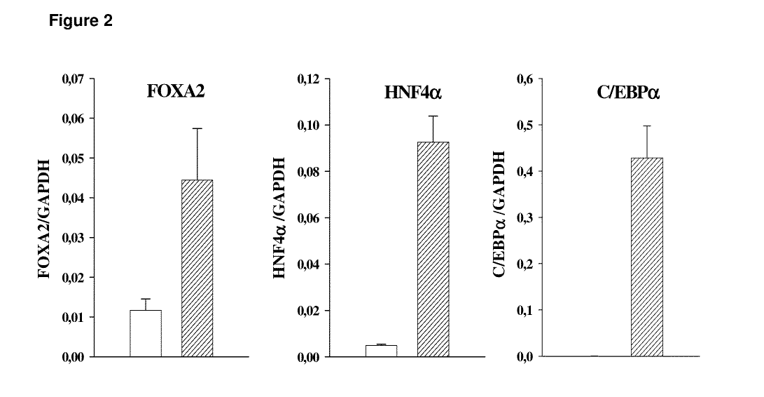

FIG. 2: mRNA expression levels relative to GAPDH mRNA expression of the transcription factors Foxa2, HNF4 and C7EBPα after lentiviral transduction of the respective cDNAs.

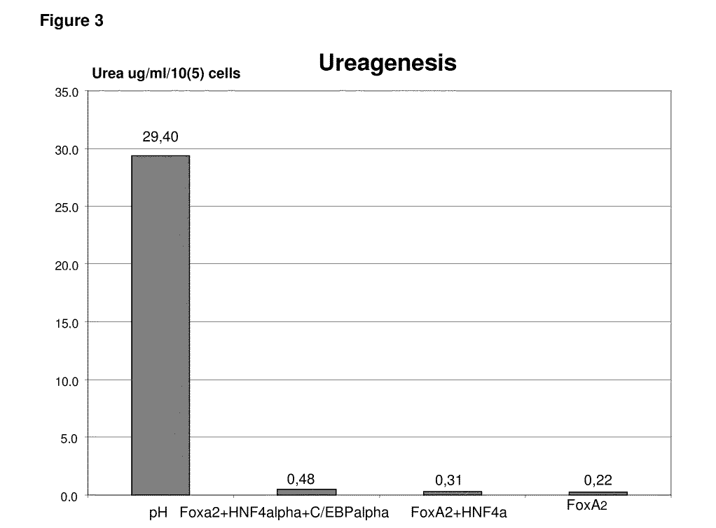

FIG. 3: Urea production in ug/m|/10(5) cells in primary hepatocytes (24 hours in culture, ADLPC with three transcription factors (Foxa2, HNF4, C/EBPα) two transcription factors (Foxa2, HNF4) and one transcription factor (FoxA2).

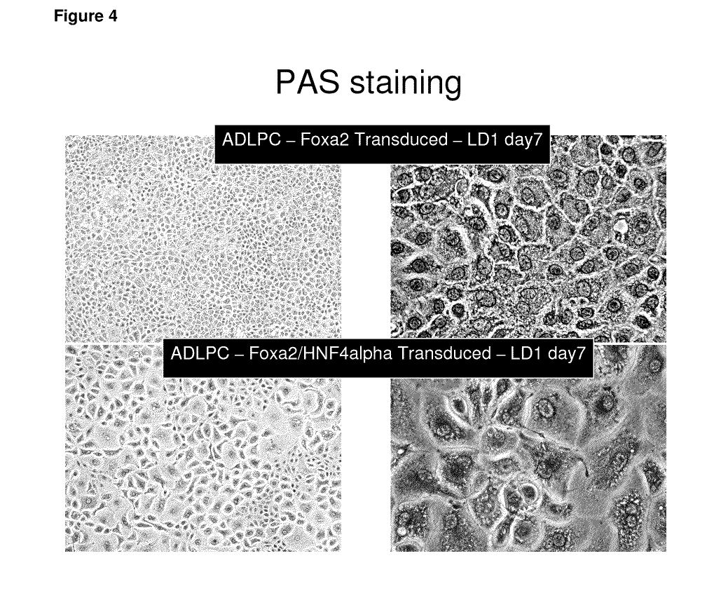

FIG. 4: PAS staining in Foxa2 transduced ADLPC cultured in LD1 medium (upper panel) and in Foxa2/HNF4 transduced ADLPC (lower panel). Left microscopic image (×200), right image (×400).

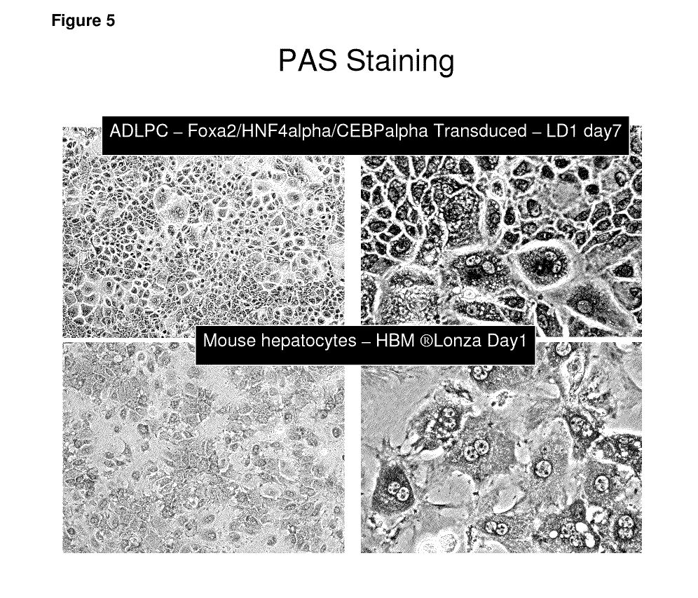

FIG. 5: PAS staining in Foxa2/HNF4/C/EBPα transduced ADLPC cultured in LD1 medium (upper panel). PAS staining in cultured primary mouse hepatocytes Left microscopic image (×200), right microscopic image (×400.)

FIG. 6: (A) Morphological appearance of ADLPC after triple transduction (phase contrast and fluorescence analysis). (B) Mock transduced ADLP.

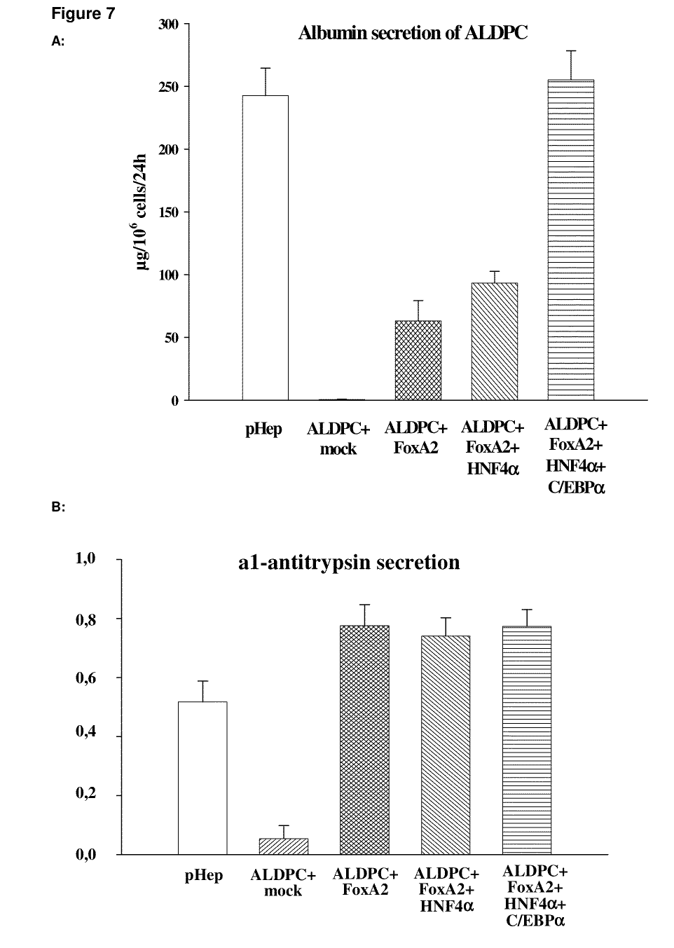

FIG. 7: (A) Concentration of mouse albumin in the culture supernatant as determined by ELISA (B) Concentration of AAT in the culture supernatant as determined by ELISA.

FIG. 8: Lentivirus plasmid with expression cassette containing HNF4 and eGFP cDNA's (upper figure) or eGFP alone (lower figure, control)

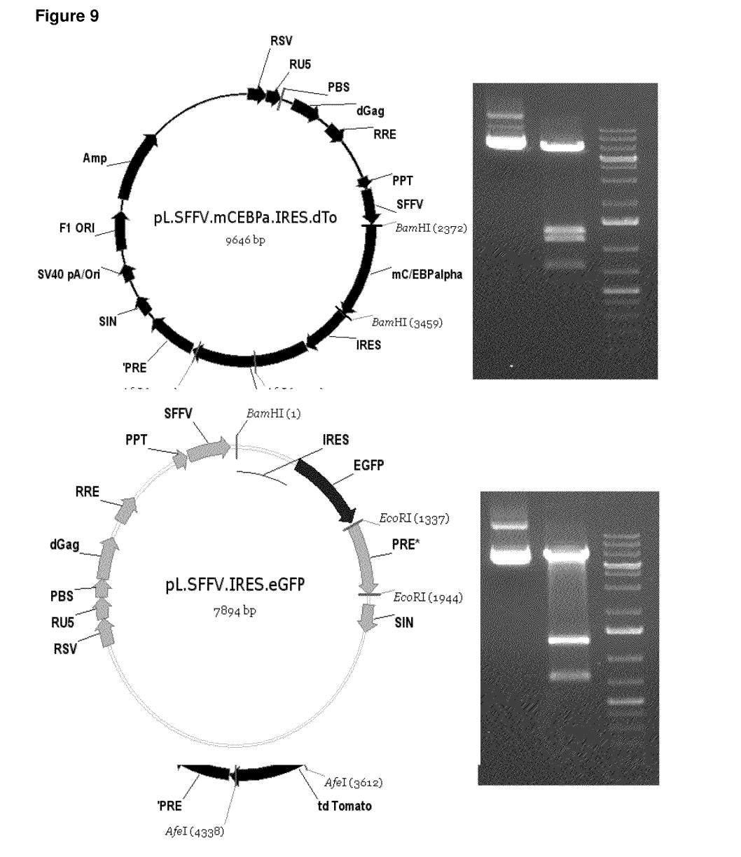

FIG. 9: Lentivirus plasmid with expression cassette containing C/EBPalpha and diTomato cDNA's (upper figure) or ditomato alone (lower figure, control)

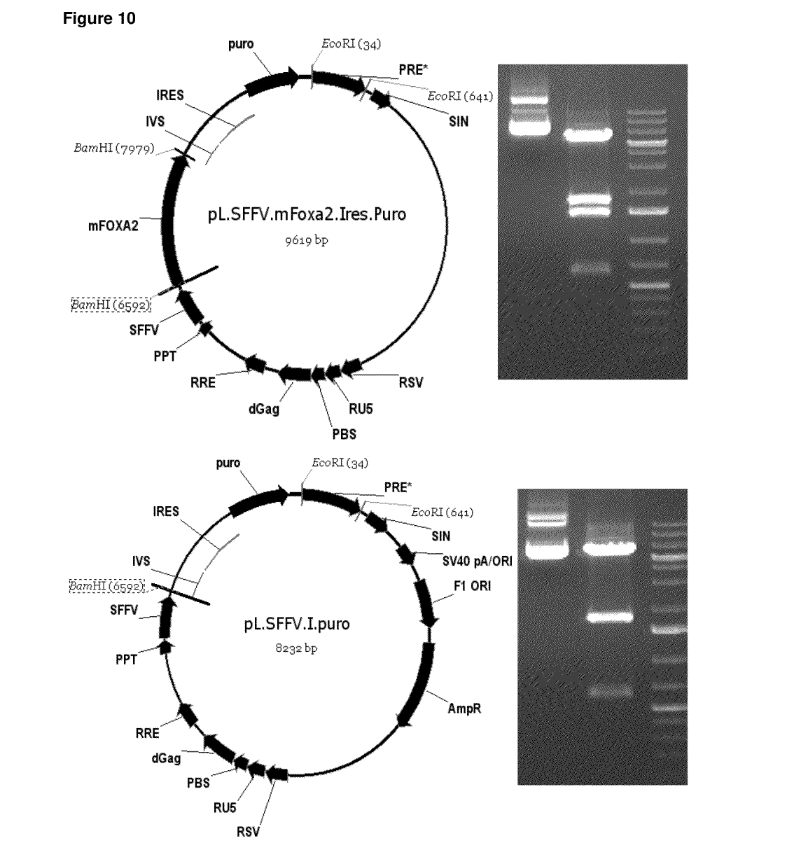

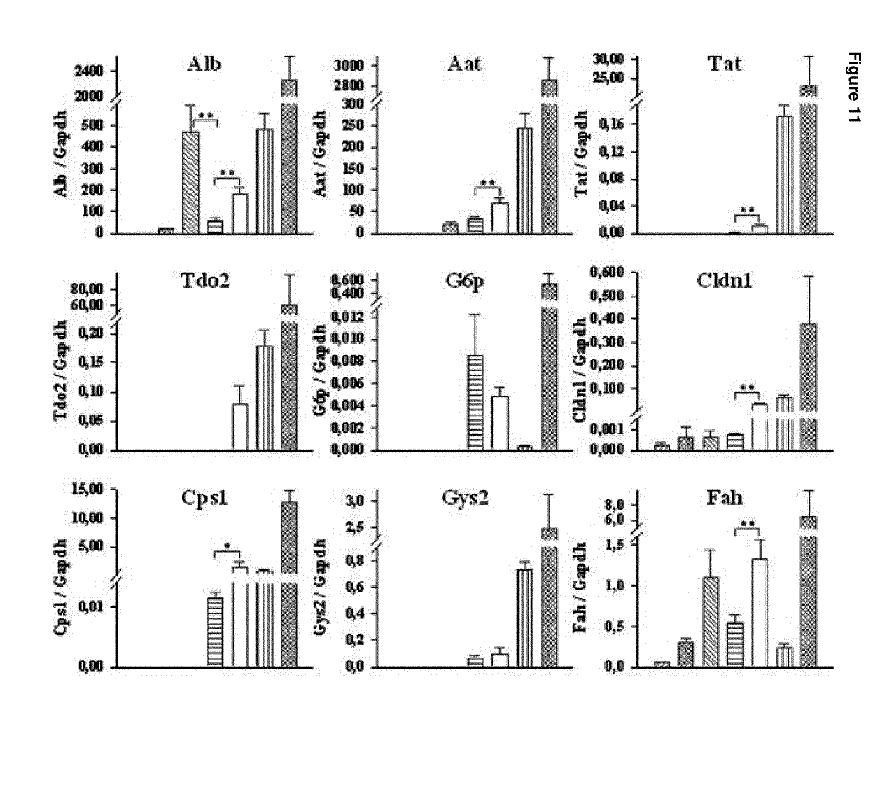

FIG. 10: Lentivirus plasmid with expression cassette containing Foxa2 and puromycin cDNA's (upper figure) or puromycin alone (lower figure, control)

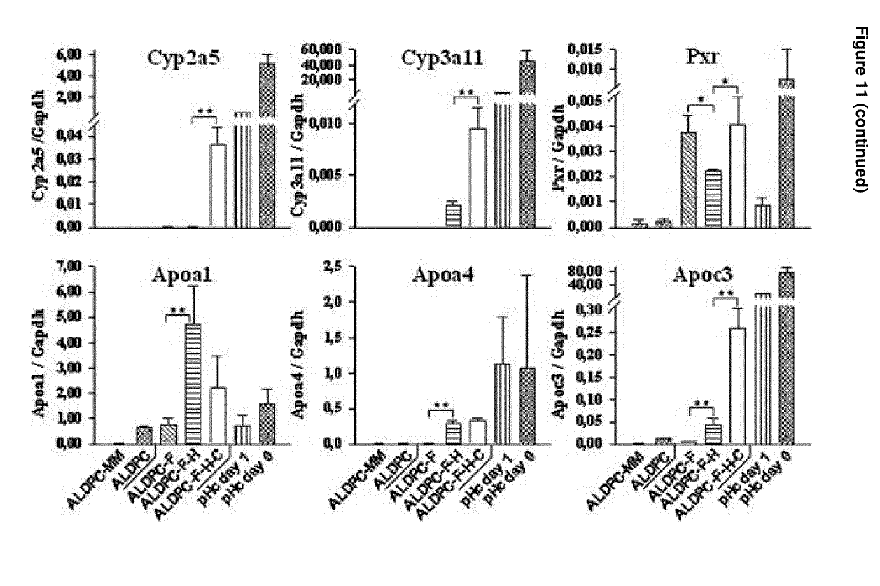

FIG. 11: mRNA expression levels of liver genes. RT-qPCR analysis for mRNA levels of the genes indicated at day 7 of the respective treatment. Bars are grouped into 1) untransduced ALDPC: ALDPC-MM, ALDPC in maintenance medium, ALDPC, ALDPC in liver differentiation medium (LD); 2) transcription factor transduced ALDPC-F, ALDPC-F-H and ALDPC-F-H-C: ALDPC in liver differentiation medium (LD), transduced with −F:Foxa2, −H:Hnf4a, −C:C/ebpa; 3) Standard: pHc day 1, primary human hepatocytes in culture (LD medium) 24 hours after isolation and pHc day 0, immediately after isolation. The data show mRNA levels normalized to Gapdh as the internal standard. Data are expressed as mean of three independent experiments±standard deviation (SD). Statistical analysis: Student Test, *P<0.05, **P<0.01.

The Examples illustrate the invention.

### EXAMPLE 1

### Experimental Procedures

### Lentivirus Vectors

Mouse transcription factor cDNA's were derived from a commercial cDNA library (Foxa2, C/EBPα from imaGenes®) and an adult mouse liver RNA sample (HNF4α), and cloned into the BamHI cloning site of a 3^{rd} generation lentiviral expression plasmid containing a SFFV promoter driven IRES-Puro (Foxa2), IRES-dTomato (C/EBPα) or IRES-eGFP (HNF4α) expression cassette. The cDNA's were re-derived from the lentivirus vectors by restriction enzyme digestion and sequenced (SeqLab®, Göttingen) for quality control before virus production.

Lentiviruses expressing the transcription factors or the respective “empty” controls were produced by transient transfection of HEK293T cells according to standard protocols. Virus containing supernatants were concentrated by centrifugation using a Centricon Plus-70 filter device (Millipore®) according to the manufacturers protocol. Virus titers were estimated by transduction of NIH3T3 cells using increments of the enriched culture supernatants.

### Transcription Factor Reporter Assays

Transcription factor reporter assays were performed using the Dual Luciferase reporter assay system according to manufacturers protocol (Promega®) For estimation of the HNF4α and C/EBPα transcriptional activities a luciferase reporter plasmid (pGL3-basic) containing a 530 bp sequence from the UGT1A9 promoter was utilized.

For the assay, 5×10⁴ HEK293T cells were seeded in 12-well plates and transfected with 1.5 ug lipofectamine (Invitrogen®) complexed DNA/well (1:1 ratio of the lentiviral transcription factor plasmid and the UGT1A9 reporter plasmid) at 70% confluency. The pRL-TK vector DNA (Promega®) expressing Renilla luciferase (19.8 ng/well) was co-transfected as internal control. All experiments were performed in triplicates and empty SFFV-Ires-eGFP and SFFV-Ires-dTomato plasmids were used as negative controls. Firefly/Renilla luminescence ratio was analyzed 36 hours after transfection in all experiments.

### Hepatocyte Isolation and Culture

Hepatocytes were isolated by a modified 2-step collagenase perfusion. The liver was first perfused with EGTA solution (5.4 mmol/L KCl, 0.44 mmol/L KH2PO4, 140 mmol/L NaCl, 0.34 mmol/L Na2HPO4.0.5 mmol/L EGTA, and 25 mmol/L Tricine, pH 7.2) with a pump rate of 8 ml/min. Subsequently liver digest medium was applied for enzymatic digestion of the tissue at 37° C. and was supplemented with Serve Collagenase NB-4 (Serve Electrophoresis GmbH, Heidelberg) at concentrations of 400-480 mg/L depending on the lot specific properties. After digestion the tissue was manually disrupted with sterile scissors and scalpels in Williams E Medium (PAN Biotech GmbH, Aidenbach). To separate undigested tissue pieces, the suspended hepatocytes were passed through a 100 μm filter into 50 ml Falcon tubes. The cell suspensions were centrifuged twice at 50 g for 5 minutes at 4° C. and the cell pellet was resuspended in Williams E Medium (PAN Biotech GmbH, Aidenbach). An aliquot of the cell preparation was separated for cell count and viability analysis (light microscopy and trypan blue exclusion test).

### Adult Mouse Liver Clonal Cell Lines

Adult liver derived progenitor cells (ALDPC) were obtained from cultured adult mouse liver cell suspensions by the “plate and wait” technique, according to published protocols (Fougere, Strick Marchand, Deschartrette 2006). The liver cell suspension was plated on a rat tail collagen I layer in hepatocyte basal medium HCM® plus supplements (Lonza, Switzerland) and 10% fetal calf serum. Twenty four hours later the medium was changed to Williams E medium containing 50 ng of epidermal growth factor (EGF) and 30 ng insulin growth factor 2 (IGF-2) and 10 ng/ml insulin. At day seven colonies of small transparent cells with an epitheloid morphology became visible. A confluent monolayer of cells was formed after three weeks of culture. After re-cloning stable and homogeneous cell cultures of ALDPC's were generated. All cell clones derived from mature mouse liver cell suspension expressed CK18 and CK 19 mRNA. For the experiments the ALDP clone E at passage 40 was used.

### Hepatic Differentiation of ALDPC's and TF Transduced ALDPC's

Hepatic differentiation was induced in adherent Clone E cells, over a period of 7 days. Cells were seeded at a density of 1.5×10⁵ cells/well in rat tail collagen-coated 6 well plates (Roche®) using two different liver differentiation media: HCM medium (Lonza®) supplemented with the standard single Quots® of insulin, hydrocortisone, transferin, antibiotics and mouse hepatocyte growth factor (HGF, 20 ng/ml) (medium LD1) or HCM medium supplemented with the standard single Quots® of insulin, transferin, antibiotics, dexamethasone (40 ng/ml), mouse HGF (20 ng/ml) and oncostatin M (20 ng/ml) (medium LD2). The same experiments were repeated in ultra low adherence culture conditions (Costar in order to allow formation of cellular aggregates.

As a first transduction step, a Foxa2 transgenic ALDPC's cell line was established by transducing passage no. 40 expanded cells with the Foxa2 expressing lentivirus and puromycine selection (2 μg/ml). After obtaining a proliferating transgenic cell population, puromycine selection was continued for 3 weeks (1 ug/ml puromycine) to assure optimal selection of transduced cells. During this step, cells were grown in Williams E media supplemented with 10% FCS, 50 ng/ml epidermal growth factor (EGF), 30 ng/ml insulin-like growth factor II (IGFII) (PeproTech®) and 10 ng/ml insulin (Sigma Aldrich®).

In a second step 1.5×10⁵ Foxa2 transgenic ALDPC's were seeded on rat tail collagen-coated 6 well plates (Roche®), cultured in liver differentiation media LD1 and co-transduced with either SFFV-HNF4alpha-IRES-eGFP and SFFV-C/EBPalpha-IRES-dTomato or with SFFV-Ires-eGFP and SFFV-Ires-dTomato control lentiviruses in adherent] culture conditions. For the transduction, a lentivirus titer of 1.8×10⁵ was used in 1 ml total transduction volume. Single transductions with SFFV-HNF4alpha-IRES-eGFP or SFFV-C/EBPalpha-IRES-dTomato have also been performed.

### Enzyme Linked Immunosorbent Assay

The amount of albumin secretion was quantified by sandwich enzyme linked immunosorbent assay using a mouse albumin ELISA quantitation kit according to manufacturers protocol (Bethyl Laboratories, Inc, TX, USA).

The mouse alpha-1 antitrypsin (mAAT) ELISA, was performed using cell culture supernatants in 96 well plates (EIA/RIA 96well, Costar) using a monoclonal antibody against murine AAT (Abcam). After incubation with a secondary HRP labelled antibody (Abcam) the substrate TMB/H₂O₂ was added. The colour development was stopped with 1N sulfuric acid after 60 min followed by photometric analysis at 450 nm and 540 nm.

### RNA Extraction and Quantitative Real Time PCR. From 24 Hours Cultured Mouse Hepatocytes and ALDPC Cells

From 24 hours cultured mouse hepatocytes and ALDPCs total RNA was extracted using the RNEasy Mini kit (Qiagen®) and subjected to reverse transcription using iScript™ cDNA synthesis kit (Biorad). The messenger RNA (mRNA) expression levels were determined by quantitative RT-PCR by a LightCycler II System (Roche Applied Science) and gene specific primers. For standardization of the qRT-PCR analysis, the PCR Fragment of each gene was cloned in the pCR4-Topo vector (Invitrogen) and sequenced. Standard curves for all genes were generated by diluting equal starting concentrations of the plasmids in a pooled sample. The concentrations of the mRNAs were calculated from these standard curves and normalized to GAPDH mRNA expression.

### Analysis of Urea Production

Ureagenic capacity of differentiated cells was assessed using the QuantiChrom™ Urea Assay kit (BioAssay Systems, CA, USA). Differentiated cells or hepatocyte cultures were incubated for 24 hours with 5 mM ammonium chloride and the amount of urea secreted into the culture medium was measured according to the manufacturers protocol. The assay utilizes a chromogenic reagent that forms a coloured complex specifically with urea. The intensity of the colour, measured at 430 nm is directly proportional to the urea concentration in the sample.

### PAS Staining

The glycogen storage capacity of the differentiated ALDPC's and control hepatocytes was assessed PAS staining. Briefly, six-well adherent cell cultures were fixed with 95% ethanol for 10 minutes, treated with periodic acid, stained with Shiffs reagent and finally counterstained with Meyer's Hemalaun solution.

### EXAMPLE 2

### Characterisation of ALDP Cells in Baseline Conditions

ALDP cells were derived from several primary liver cell cultures in medium containing EGF. IGF2 and insulin. A tightly packed monolayer of cells with epitheloid morphology was formed after three to six weeks of culture. Analysis of several ALDP clones showed uniform expression of cytokeratins 7, 8, 18 and 19 mRNA's, E- and N-Cadherin, low levels of liver enriched transcription factor mRNA's including HNF-1α, Foxa2 and HNF4α and no detectable concentrations of C/EBPα mRNA. Only trace amounts of albumin, α1-antitrypsin and α-fetoprotein were secreted into the supernatant. From the twelve mRNA's, which were chosen for differential expression in mouse hepatocytes only the PXR mRNA was detectable at a range close to primary hepatocytes.

### EXAMPLE 3

### Hepatic Differentiation of ALDP Cells

The ALDP's, also named BAML or LSC's by others, have been proposed as liver progenitor cells. First tested was the hepatic differentiation capacity in the presence of factors and conditions frequently utilized for unidirectional differentiation to hepatocytes by creating 4 different differentiation settings as described in the methods section: two liver differentiation media (LD1 and LD2) were assessed using adherent or ultra-low adherent culture conditions.

Compared to baseline conditions no significant differences in secreted albumin, AFP and α1-AAT levels were detected after an induction period of 7 days. Glucose-6 phosphatase and Apolipoprotein A1 mRNA's were strongly induced in all four differentiation conditions (p<0.001 compared to baseline). Alb, AAT, TAT, TDO, ApoA4 mRNA's, which were not detectable in baseline culture conditions, were induced by the various differentiation conditions at low levels compared to primary hepatocytes. No mRNA induction was observed for the Cytochrom p450 genes. Claudin and ApoC3 indicating an only limited and partial hepatic differentiation potential with conventional protocols. The data did not clearly favour one of the four differentiation protocols applied. Subsequent experiments were thus performed in LD1 medium in adherent cell cultures.

### EXAMPLE 4

### Hepatic Differentiation of ALDPC's by Transcription Factors

The concept of forward programming by sequential expression of defined transcription factors was tested in Foxa2 transgenic ALDP cells by transduction of LV vectors constitutively expressing murine HNF4α-IRES-eGFP and C/EBPα-IRES-dTomato cDNA's. At the end of the differentiation protocol, flow cytometry analysis has indicated 87% eGFP positive, 60% dTomato positive and 34% double positive cells (mean of a set of 3 independent transduction experiments). Cells transduced with each lentivirus vector significantly showed increased mRNA levels of the respective transcription factors (Fig XY).

Compared to the parental cells albumin secretion levels increased stepwise in FOXA2 (single), FOXA2/HNF4α (double) and in FOXA2/HNF4α/C/EBPα (triple) transduced ALDPC's. The triple transduced cells secreted albumin at levels not statistically different from the concentrations measured in supernatants of 24 h cultured primary hepatocytes. In contrast, AAT protein secretion reached mature hepatocyte levels already in double transduced cells and was not further stimulated in the triple transduced cell population. (In the figure it looks like there is no difference between single, double and triple transduction)

All of the 12 genes representing a wide range of hepatocyte functions were induced in triple transduced cells. The mRNA's of CYP3A11 and ApoC3, although significantly (p<0.001) induced compared to levels of non-transduced ALDP cells, did not reach the concentrations of mature hepatocytes. The quantities of gene expression relative to GAPDH expression exceeded those of mouse iPS derived hepatocyte like cells for 11 of the 12 genes with the exception of TAT mRNA expression levels.

### EXAMPLE 5

### Glycogene Storage

Storage of glycogen is a characteristic feature of mature hepatocytes. Thus primary mouse hepatocytes. ALDP cells and lentivirus transduced ALDP cells were stained with PAS. Non transduced or Foxa2 transgenic ALDP cells showed inconsistent or only very faint PAS positive staining. Double transduced or triple transduced cells, however, presented a much stronger PAS positive staining, comparable adult day 1 cultured hepatocytes. Particularly, the double nucleated cells closely resembling adult hepatocytes by morphology, presented also the brightest positive staining.

### EXAMPLE 6

### Ureagenesis

Ureagenesis was not detectable in non transduced ALDP cells but detectable in transduced cells at the end of differentiation protocol. Triple transduced cells showed the best ureagenic ability, however, a 60 fold lower urea production was noted, compared to day 1 adult hepatocytes.

### REFERENCES

Takahashi K, Yamanaka S. Induction of pluripotent stem cells from mouse embryonic and adult fibroblast cultures by defined factors. Cell 2006; 126:663-76.
Takahashi K, Tanabe K, Ohnuki M, Narita M, Ichisaka T, Tomoda K, Yamanaka S. Induction of pluripotent stem cells from adult human fibroblasts by defined factors. Cell 2007; 131:861-72.
Kyrmizi I, Hatzis P, Katrakili N, Tronche F, Gonzalez F J, Talianidis I. Plasticity and expanding complexity of the hepatic transcription factor network during liver development. Genes Dev. 2006 Aug. 15; 20(16):2293-305.
Lemaigre F P. Mechanisms of liver development: concepts for understanding liver disorders and design of novel therapies. Gastroenterology. 2009 July; 137(1):62-79.
Wederell E D, Bilenky M, Cullum R, Thiessen N, Dagpinar M, Delaney A, Varhol R, Zhao Y, Zeng T, Bernier B, Ingham M, Hirst M, Robertson G, Marra M A, Jones S, Hoodless P A. Global analysis of in vivo Foxa2-binding sites in mouse adult liver using massively parallel sequencing. Nucleic Acids Res. 2008 August; 36(14):4549-64.
Zaret K S, Grompe M. Generation and regeneration of cells of the liver and pancreas. Science. 2008 Dec. 5; 322(5907):1490-4.
Tan E H, Ma F J, Gopinadhan S, Sakban R B, Wang N D. C/EBP alpha knock-in hepatocytes exhibit increased albumin secretion and urea production. Cell Tissue Res. 2007 December; 330(3):427-35.
Nerlov C. The C/EBP family of transcription factors: a paradigm for interaction between gene expression and proliferation control. Trends Cell Biol. 2007 July; 17(7):318-24.
Tseng H H, Hwang Y H, Yeh K T, Chang J G, Chen Y L, Yu H S. Reduced expression of C/EBP alpha protein in hepatocellular carcinoma is associated with advanced tumor stage and shortened patient survival. J Cancer Res Clin Oncol. 2009 February; 135(2):241-7.
Parviz F, Matullo C. Garrison W D, Savatski L. Adamson J W, Ning G. Kaestner K H, et al. Hepatocyte nuclear factor 4 alpha controls the development of a hepatic epithelium and liver morphogenesis. Nat Genet 2003; 34: 292-296.
Li J, Ning G, Duncan S A. Mammalian hepatocyte differentiation requires the transcription factor HNF-4alpha. Genes Dev 2000; 14: 464-474.
Hayhurst G P, Lee Y H, Lambert G, Ward J M, Gonzalez F J. Hepatocyte nuclear factor 4alpha (nuclear receptor 2A1) is essential for maintenance of hepatic gene expression and lipid homeostasis. Mol Cell Biol 2001; 21: 1393-1403.
Kang X Q, Zang W J, Bao L J, Li D L, Song T S, Xu X L, et al. Fibroblast growth factor-4 and hepatocyte growth factor induce differentiation of human umbilical cord blood-derived mesenchymal stem cells into hepatocytes. World J Gastroenterol. 2005; 11:7461-7465.
Ong S Y, Dai H, Leong K W. Hepatic differentiation potential of commercially available human mesenchymal stem cells. Tissue Eng. 2006; 12:3477-85.
Sgodda M, Aurich H, Kleist S, Aurich I, König S, Dollinger M M, et al. Hepatocyte differentiation of mesenchymal stem cells from rat peritoneal adipose tissue in vitro and in vivo. Exp Cell Res. 2007; 313:2875-2886.
Vierbuchen T, Ostermeier A, Pang Z P, Kokubu Y, Südhof T C, Wernig M. Direct conversion of fibroblasts to functional neurons by defined factors. Nature. 2010 Feb. 25; 463(7284):1035-41.
Wünsch E. & Heidrich H. C. Physiol. Chemie (1963), 333:149-159
Yamazaki S, Miki K, Hasegawa K, Sata M, Takayama T, Makuuchi M. Sera from liver failure patients and a demethylating agent stimulate transdifferentiation of murine bone marrow cells into hepatocytes in coculture with nonparenchymal liver cells. J Hepatol. 2003; 39:17-23.
Ishii K, Yoshida Y, Akechi Y, Sakabe T, Nishio R, Ikeda R, Terabayashi K, Matsumi Y, Gonda K, Okamoto H, Takubo K, Tajima F, Tsuchiya H, Hoshikawa Y, Kurimasa A, Umezawa A, Shiota G. Hepatic differentiation of human bone marrow-derived mesenchymal stem cells by tetracycline-regulated hepatocyte nuclear factor 3beta. Hepatology. 2008 August; 48(2):597-606.
Inoue Y, Inoue J, Lambert G, Yim S H, Gonzalez F J. Disruption of hepatic C/EBPalpha results in impaired glucose tolerance and age-dependent hepatosteatosis. J Biol Chem. 2004 Oct. 22; 279(43):44740-8.
Wang X, Huang G, Mei S, Qian J, Ji J, Zhang J. Over-expression of C/EBP-alpha induces apoptosis in cultured rat hepatic stellate cells depending on p53 and peroxisome proliferator-activated receptor-gamma. Biochem Biophys Res Commun. 2009 Mar. 6; 380(2):286-91.
Ishizaka S, Shiroi A, Kanda S, Yoshikawa M, Tsujinoue H, Kuriyama S, Hasuma T, Nakatani K, Takahashi K. Development of hepatocytes from ES cells after transfection with the HNF-3beta gene. FASEB J. 2002 September; 16(11):1444-6
Kubo A, Kim Y H, Irian S, Kasuda S, Takeuchi M, Ohashi K, Iwano M, Dohi Y, Saito Y, Snodgrass R, Keller G. The homeobox gene Hex regulates hepatocyte differentiation from embryonic stem cell-derived endoderm. Hepatology. 2010 February; 51(2):633-41.

1. A method for producing cells having a mature hepatocyte phenotype, comprising:
(a) providing a cell population comprising precursor cells of mature hepatocytes, wherein said cell population is obtained from a subject or derived from a cell line; and
(b) introducing into said precursor cells a group of differentiation factors and/or the nucleic acid sequences encoding said differentiation factors, said group consisting of
(i) one or more member(s) of the Foxa subfamily,
(ii) HNF-4α and
(iii) C/EBPα
thereby differentiating said precursor cells into cells having a mature hepatocyte phenotype.

2. The method according to claim 1, further comprising:
(c) isolating cells having a mature hepatocyte phenotype from the cell population obtained in step (b).

3. The method of claim 1 or 2, wherein said introducing into said precursor cells is effected by
(i) transforming said precursor cells with one or more nucleic acids molecule(s) comprising the nucleic acid sequences encoding
(i) one or more member(s) of the Foxa subfamily,
(ii) HNF-4α and
(iii) C/EBPα
in an expressible form; and/or
(ii) microinjection, electroporation, lipofection and/or protein transduction of
(i) one or more member(s) of the Foxa subfamily,
(ii) HNF-4α and
(iii) C/EBPα
into said precursor cells.

4. The method of claim 3, wherein the one or more nucleic acids molecule(s) are one or more vector(s).

5. The method of claim 4, wherein the one or more vector(s) are one or more lentiviral vector(s).

6. The method of any one of claims 3 to 5, wherein the nuclei acid sequences encode (i) the one or more member(s) of the Foxa subfamily, (ii) HNF-4α and (iii) C/EBPα in an inducible expressible form.

7. The method of any one of claims 1 to 6, wherein the member of the Foxa subfamily is Foxa2.

8. The method of any one of claims 1 to 7, wherein the precursor cells of mature hepatocytes are embryonic stem cells, adult stem cells, or iPS cells.

9. The method of any one of claims 1 to 7, wherein the precursor cells of mature hepatocytes are
(a) primitive epithelial progenitor cells obtained from liver suspension, or
(b) adult liver derived progenitor cells.

10. The method of any of one of claims 1 to 9, wherein the obtained cells are free of pathogens.

11. The method of any one of claims 1 to 10, wherein the cells having a mature hepatocyte phenotype are characterized by the expression of albumin, AAT, TAT, TDO, G6P, Cyp3A7, Cyp2A5, PXR, Apo44, ApoC3 and ApoA1.

12. The method of any one of claims 1 to 11, wherein the cells having a mature hepatocyte phenotype are characterized by the capability to store glycogen intracellularly and the capability to generate urea.

13. The method of any one of claims 1 to 12, wherein the cells having a mature hepatocyte phenotype are characterized by comprising cells with dispersed chromatin and/or two or more cell nuclei.

14. The method of any one of claims 1 to 13, wherein the differentiation step (b) is carried out in the presence of one or more additional differentiation factors selected from the group consisting of histone deacetylase inhibitors.

15. The cell obtained by the method according to any one of claims 1 to 14 for use in treating or preventing a liver disease.

16. A method for identifying a compound having an pharmacological, cytotoxic, proliferative, transforming or differentiating effect on cells having a mature hepatocyte phenotype obtained by the method according to any one of claims 1 to 14, comprising:
(a) contacting said cells having a mature hepatocyte phenotype with a test compound; and
(b) determining whether the test compound has one or more of said effects on said cells having a mature hepatocyte phenotype.
